(19) United States
(12) Patent Application Publication
(10) Pub. No.: US 2026/0216176 A1
(43) Pub. Date: Jul. 30, 2026

(54) USE OF PCLX-001 OR PCLX-002 AS A RADIOSENSITIZER

(71) Applicant: ACYLEX PHARMACEUTICALS INC., Calgary (CA)

(72) Inventor: Deepak Karthesh DINAKARAN, Derwood, MD (US)

(21) Appl. No.: 19/146,289

(22) PCT Filed: Jan. 11, 2024
(86) PCT No.: PCT/CA2024/050030

§ 371 Date: Jul. 8, 2025

Related U.S. Application Data
(60) Provisional application No. 63/438,307, filed on Jan. 11, 2023.
(60) Provisional application No. 63/531,839, filed on Aug. 10, 2023.

(51) Int. Cl. A61K 31/496; A61K 41/00; A61K 45/06; A61P 35/00

(52) U.S. Cl. CPC A61K 31/496; A61K 41/0038; A61K 45/06; A61P 35/00

(57) ABSTRACT

Described herein is the use of PCLX-001, PCLX-002 or pharmaceutically acceptable salts thereof as a radiosensitizer of cancerous cells in a subject in need of radiation therapy. The radio sensitizer is applied to a subject with cancer undergoing radiotherapy, especially cancers of the brain, such as astrocytoma, glioma, embryonal tumour, non-malignant brain tumor, pediatric brain tumour, or metastatic tumours. The radiosensitizer is administered at dosing amounts to achieve blood, target organ or tumor drug concentrations in the range of about 50 nM to about 150 nM.

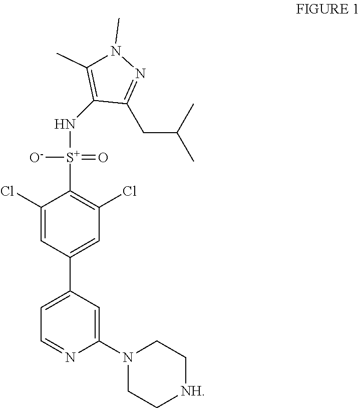 [SMILES: Cc1c(N[S+](=O)([O-])c2c(Cl)cc(-c3ccnc(N4CCNCC4)c3)cc2Cl)c(CC(C)C)nn1C]

### FIELD

The present disclosure relates generally to use of PCLX-001 or PCLX-002 as a radiosensitizer.

### BACKGROUND

About half of patients with cancer are treated with radiation therapy, either alone or in combination with other types of cancer treatment.

There remains a need for radiosensitizers in the treatment of subjects with cancer.

### SUMMARY

In one aspect there is provided a method of radiosensitizing a cancerous cell in a subject in need of radiation therapy, comprising: administering to a subject a compound or PCLX-001, or pharmaceutically acceptable salt thereof.

In one aspect there is provided a method of radiosensitizing a cancerous cell in a subject in need of radiation therapy, comprising: administering to a subject a compound of PCLX-001, or pharmaceutically acceptable salt thereof.

In one example, said radiation therapy is external radiation therapy, internal radiation therapy or systemic radiation therapy.

In one example, wherein said subject has cancer of the bladder, brain, breast, cervix, larynx, lung, prostate, vagina, thyroid, pancreas, ovary, breast, uterus, gallbladder, perianal and pelvic regions; colorectal cancers; gynecological cancers; cancer of the small intestine; small cell lung cancer; head and neck cancer; bronchial cancer; oral cancer; rectal cancer; tracheal cancer; or adult non-Hodgkin lymphoma.

In one example, wherein said subject is a human.

In one aspect there is provided a method of treating a subject with cancer, suspected of having a cancer, or at risk of developing a cancer, comprising administering a radiosensitizer, wherein the radiosensitizer is PCLX-001, and administering radiation therapy.

In one example, wherein said radiation therapy is external radiation therapy, internal radiation therapy or systemic radiation therapy.

In one example, wherein the said subject has cancer of the bladder, brain, breast, cervix, larynx, lung, prostate, vagina, thyroid, pancreas, ovary, breast, uterus, gallbladder, perianal and pelvic regions; colorectal cancers; gynecological cancers; cancer of the small intestine; small cell lung cancer; head and neck cancer; bronchial cancer; oral cancer; rectal cancer; tracheal cancer; or adult non-Hodgkin lymphoma.

In one example, wherein the brain cancer is an astrocytoma, glioma, embryonal tumour, non-malignant brain tumor, pediatric brain tumour, or metastatic tumours.

In one example, wherein the astrocytoma is anaplastic astrocytoma, diffuse astrocytoma, pilocytic astrocytoma, or glioblastoma.

In one example, wherein the glioma is diffuse midline glioma, oligodendroglioma, ependymoma, or optic pathway glioma.

In one example, wherein the embryonal tumour is atypical teratoid/rhaboid (AT/RT), or medulloblastoma.

In one example, wherein the non-malignant brain tumour is acoustic neuroma, meningioma, pituitary adenomas, craniopharyngioma, or pilocytic astrocytoma.

In one example, wherein the pediatric brain tumour is atypical teratoid/rhaboid tumour (AT/RT), diffuse midline glioma, medulloblastoma, pilocytic astrocytoma, craniopharyngioma, ependymoma, or optic pathway glioma.

In one example, further comprising administering one or more drugs for treating brain cancer comprising conjugates of methotrexate, vinca alkaloids, carmustine, cisplatin, carboplatin, nitrosourea, hydroxyurea, cyclophosphamide, etoposide, procarbazine, irinotecan, lomustine, vincristine, and/or temozolomide.

In one example, wherein said subject is a human.

As described herein, there is also provided the following embodiments:
1. Use of PCLX-001, or pharmaceutically acceptable salt thereof, for radiosensitizing a cancerous cell in a subject in need of radiation therapy.
2. Use of PCLX-001, or pharmaceutically acceptable salt thereof, in the manufacture of a medicament for radiosensitizing a cancerous cell in a subject in need of radiation therapy.
3. The use of embodiment 1 or 2, wherein said radiation therapy is external radiation therapy, internal radiation therapy or systemic radiation therapy.
4. The use of any one of embodiments 1 to 3, wherein said subject has cancer of the bladder, brain, breast, cervix, larynx, lung, prostate, vagina, thyroid, pancreas, ovary, breast, uterus, gallbladder, perianal and pelvic regions; colorectal cancers; gynecological cancers; cancer of the small intestine; small cell lung cancer; head and neck cancer; bronchial cancer; oral cancer; rectal cancer; tracheal cancer; or adult non-Hodgkin lymphoma.
5. The use of embodiment 4, wherein the brain cancer is an astrocytoma, glioma, embryonal tumour, non-malignant brain tumor, pediatric brain tumour, or metastatic tumours.
6. The use of embodiment 5, wherein the astrocytoma is anaplastic astrocytoma, diffuse astrocytoma, pilocytic astrocytoma, or glioblastoma.
7. The use of embodiment 5, wherein the glioma is diffuse midline glioma, oligodendroglioma, ependymoma, or optic pathway glioma.
8. The use of embodiment 5, wherein the embryonal tumour is atypical teratoid/rhaboid (AT/RT), or medulloblastoma.
9. The use of embodiment 5, wherein the non-malignant brain tumour is acoustic neuroma, meningioma, pituitary adenomas, craniopharyngioma, or pilocytic astrocytoma.
10. The use of embodiment 5, wherein the pediatric brain tumour is atypical teratoid/rhaboid tumour (AT/RT), diffuse midline glioma, medulloblastoma, pilocytic astrocytoma, craniopharyngioma, ependymoma, or optic pathway glioma.
11. The use of any one of embodiments 1 to 10, wherein said subject is a human.
12. The use of any one of embodiments 1 to 11, wherein the compound PCLX-001 or pharmaceutiall acceptable salt thereof is at a dosing range of about 50 nM to about 150 nM.
13. Use of a radiosensitizer, wherein the radiosensitizer is PCLX-001, and use of radiation therapy, for treating a subject with cancer, suspected of having a cancer, or at risk of developing a cancer.
14. Use of a radiosensitizer, wherein the radiosensitizer is PCLX-001, in the manufacture of a medicament, and and use of radiation therapy, for treating a subject with cancer, suspected of having a cancer, or at risk of developing a cancer.
15. The use of embodiment 13, or 14 wherein said radiation therapy is external radiation therapy, internal radiation therapy or systemic radiation therapy.
16. The use of any one of embodiments 13 to 15, wherein the said subject has cancer of the bladder, brain, breast, cervix, larynx, lung, prostate, vagina, thyroid, pancreas, ovary, breast, uterus, gallbladder, perianal and pelvic regions; colorectal cancers; gynecological cancers; cancer of the small intestine; small cell lung cancer; head and neck cancer; bronchial cancer; oral cancer; rectal cancer; tracheal cancer; or adult non-Hodgkin lymphoma.
17. The use of embodiment 16, wherein the brain cancer is an astrocytoma, glioma, embryonal tumour, non-malignant brain tumor, pediatric brain tumour, or metastatic tumours.
18. The use of embodiment 17, wherein the astrocytoma is anaplastic astrocytoma, diffuse astrocytoma, pilocytic astrocytoma, or glioblastoma.
19. The use of embodiment 17, wherein the glioma is diffuse midline glioma, oligodendroglioma, ependymoma, or optic pathway glioma.
20. The use of embodiment 17, wherein the embryonal tumour is atypical teratoid/rhaboid (AT/RT), or medulloblastoma.
21. The use of embodiment 17, wherein the non-malignant brain tumour is acoustic neuroma, meningioma, pituitary adenomas, craniopharyngioma, or pilocytic astrocytoma.
22. The use of embodiment 17, wherein the pediatric brain tumour is atypical teratoid/rhaboid tumour (AT/RT), diffuse midline glioma, medulloblastoma, pilocytic astrocytoma, craniopharyngioma, ependymoma, or optic pathway glioma.
23. The use of any one of embodiments 13 to 22, further comprising use or one or more drugs for treating brain cancer comprising: conjugates of methotrexate, vinca alkaloids, carmustine, cisplatin, carboplatin, nitrosourea, hydroxyurea, cyclophosphamide, etoposide, procarbazine, irinotecan, lomustine, vincristine, and/or temozolomide.
24. The use of any one of embodiments 13 to 22, further comprising use of one or more drugs in the manufacture of a medicament for treating brain cancer comprising: conjugates of methotrexate, vinca alkaloids, carmustine, cisplatin, carboplatin, nitrosourea, hydroxyurea, cyclophosphamide, etoposide, procarbazine, irinotecan, lomustine, vincristine, and/or temozolomide.
25. The use of any one of embodiments 13 to 24, wherein said subject is a human.
26. The use of any one of embodiments 13 to 23, comprising use of the radiosensitizer at a dosing to achieve blood or target organ or tumor drug concentrations in the range of about 50 nM to about 150 nM.
27. The use of any one of embodiments 13 to 26, comprising use of the radiosensitizer at Time A and use of radiation therapy at Time B for achieving a target dosing range in blood or target organ or tumor drug concentration of about 50 nM to about 150 nM at the cancer site, or at the potential cancer site.
28. The use of embodiment 27, wherein Time B is about 2 hours to about 4 hours after Time A when administering the radiosensitizer at Time A comprises orally administering the radiosensitizer.
29. The use of embodiment 27, wherein Time B is about 72 hours after Time A when administering the radiosensitizer at Time A comprises commencing radiosensitizer therapy.
30. The use of any one of embodiments 1 to 29, wherein administering PCLX-001 comprises PCLX-001 crossing the blood brain barrier.
31. The use of embodiment 30, wherein use of PCLX-001 comprises PCLX-001 crossing the blood brain barrier and acting as a radiosensitizer for primary brain tumors.
32. The use of embodiment 30 or 31, wherein use of PCLX-001 comprises PCLX-001 crossing the blood brain barrier and acting as a radiosensitizer for primary brain tumors about 4 hours after administration.
33. The use of any one of embodiments 30 to 32, wherein use of PCLX-001 comprises PCLX-001 crossing the blood brain barrier and acting as a radiosensitizer for secondary brain tumors.
34. The use of any one of embodiments 30 to 33, wherein use of PCLX-001 comprises PCLX-001 crossing the blood brain barrier and acting as a radiosensitizer for secondary brain tumors about 4 hours after administration.
35. The use of any one of embodiments 13 to 34, wherein the patient discontinues use of a proton pump inhibitor prior to treatment with the radiosensitizer and radiation therapy, if the patient is using the proton pump inhibitor.
36. A method of radiosensitizing a cancerous cell in a subject in need of radiation therapy, comprising: administering to a subject compound PCLX-001, or pharmaceutically acceptable salt thereof.
37. The method of embodiment 36, wherein said radiation therapy is external radiation therapy, internal radiation therapy or systemic radiation therapy.
38. The method of embodiment 36 or 37, wherein said subject has cancer of the bladder, brain, breast, cervix, larynx, lung, prostate, vagina, thyroid, pancreas, ovary, breast, uterus, gallbladder, perianal and pelvic regions; colorectal cancers; gynecological cancers; cancer of the small intestine; small cell lung cancer; head and neck cancer; bronchial cancer; oral cancer; rectal cancer; tracheal cancer; or adult non-Hodgkin lymphoma.
39. The method of embodiment 38, wherein the brain cancer is an astrocytoma, glioma, embryonal tumour, non-malignant brain tumor, pediatric brain tumour, or metastatic tumours.
40. The method of embodiment 39, wherein the astrocytoma is anaplastic astrocytoma, diffuse astrocytoma, pilocytic astrocytoma, or glioblastoma.
41. The method of embodiment 39, wherein the glioma is diffuse midline glioma, oligodendroglioma, ependymoma, or optic pathway glioma.
42. The method of embodiment 39, wherein the embryonal tumour is atypical teratoid/rhaboid (AT/RT), or medulloblastoma.
43. The method of embodiment 39, wherein the non-malignant brain tumour is acoustic neuroma, meningioma, pituitary adenomas, craniopharyngioma, or pilocytic astrocytoma.
44. The method of embodiment 39, wherein the pediatric brain tumour is atypical teratoid/rhaboid tumour (AT/RT), diffuse midline glioma, medulloblastoma, pilocytic astrocytoma, craniopharyngioma, ependymoma, or optic pathway glioma.
45. The method of any one of embodiments 36 to 44, wherein said subject is a human.
46. The method of any one of embodiments 36 to 45, comprising administering the compound PCLX-001 at a dosing range of about 50 nM to about 150 nM.
47. A method of treating a subject with cancer, suspected of having a cancer, or at risk of developing a cancer, comprising administering a radiosensitizer, wherein the radiosensitizer is PCLX-001, and administering radiation therapy.
48. The method of embodiment 47, wherein said radiation therapy is external radiation therapy, internal radiation therapy or systemic radiation therapy.
49. The method of embodiment 47 or 48, wherein the said subject has cancer of the bladder, brain, breast, cervix, larynx, lung, prostate, vagina, thyroid, pancreas, ovary, breast, uterus, gallbladder, perianal and pelvic regions; colorectal cancers; gynecological cancers; cancer of the small intestine; small cell lung cancer; head and neck cancer; bronchial cancer; oral cancer; rectal cancer; tracheal cancer; or adult non-Hodgkin lymphoma.
50. The method of embodiment 49, wherein the brain cancer is an astrocytoma, glioma, embryonal tumour, non-malignant brain tumor, pediatric brain tumour, or metastatic tumours.
51. The method of embodiment 50, wherein the astrocytoma is anaplastic astrocytoma, diffuse astrocytoma, pilocytic astrocytoma, or glioblastoma.
52. The method of embodiment 50, wherein the glioma is diffuse midline glioma, oligodendroglioma, ependymoma, or optic pathway glioma.
53. The method of embodiment 50, wherein the embryonal tumour is atypical teratoid/rhaboid (AT/RT), or medulloblastoma.
54. The method of embodiment 50, wherein the non-malignant brain tumour is acoustic neuroma, meningioma, pituitary adenomas, craniopharyngioma, or pilocytic astrocytoma.
55. The method of embodiment 50, wherein the pediatric brain tumour is atypical teratoid/rhaboid tumour (AT/RT), diffuse midline glioma, medulloblastoma, pilocytic astrocytoma, craniopharyngioma, ependymoma, or optic pathway glioma.
56. The method of any one of embodiments 48 to 55, further comprising administering one or more drugs for treating brain cancer comprising: conjugates of methotrexate, vinca alkaloids, carmustine, cisplatin, carboplatin, nitrosourea, hydroxyurea, cyclophosphamide, etoposide, procarbazine, irinotecan, lomustine, vincristine, and/or temozolomide.
57. The method of any one of embodiments 48 to 56, wherein said subject is a human.
58. The method of any one of embodiments 48 to 57, comprising administering the radiosensitizer at a dosing to achieve blood or target organ or tumor drug concentrations in the range of about 50 nM to about 150 nM.
59. The method of any one of embodiments 48 to 58, comprising administering the radiosensitizer at Time A and administering radiation therapy at Time B for achieving a target dosing range in blood or target organ or tumor drug concentration of about 50 nM to about 150 nM at the cancer site, or at the potential cancer site.
60. The method of embodiment 59, wherein Time B is about 2 hours to about 4 hours after Time A when administering the radiosensitizer at Time A comprises orally administering the radiosensitizer.
61. The method of embodiment 59, wherein Time B is about 72 hours after Time A when administering the radiosensitizer at Time A comprises commencing radiosensitizer therapy.
62. The method of any one of embodiments 36 to 61, wherein administering PCLX-001 comprises PCLX-001 crossing the blood brain barrier.
63. The method of embodiment 62, wherein administering PCLX-001 comprises PCLX-001 crossing the blood brain barrier and acting as a radiosensitizer for primary brain tumors.
64. The method of embodiment 62 or 63, wherein administering PCLX-001 comprises PCLX-001 crossing the blood brain barrier and acting as a radiosensitizer for primary brain tumors about 4 hours after administration.
65. The method of any one of embodiments 62 to 64, wherein administering PCLX-001 comprises PCLX-001 crossing the blood brain barrier and acting as a radiosensitizer for secondary brain tumors.
66. The method of any one of embodiments 26 to 65, wherein administering PCLX-001 comprises PCLX-001 crossing the blood brain barrier and acting as a radiosensitizer for secondary brain tumors about 4 hours after administration.
67. The method of any one of embodiments 47 to 66, wherein the patient discontinues use of a proton pump inhibitor prior to treatment with the radiosensitizer and radiation therapy, if the patient is using the proton pump inhibitor.

### BRIEF DESCRIPTION OF THE FIGURES

Embodiments of the present disclosure will now be described, by way of example only, with reference to the attached Figures.

FIG. 1 depicts the structure of PCLX-001.

FIG. 2 depicts the structure of PCLX-002.

FIG. 3 depicts an Alamar blue-based colorimetric assay measuring cell viability and survival of U251 glioblastoma cell line in response to radiotherapy and gradient of PCLX-001 drug concentration. Drug was given 72 hours prior to irradiation, and viability was assayed 72 hours post-irradiation.

### DETAILED DESCRIPTION

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

As will be described in more detail below, in some aspects, there is described compounds, compositions, methods and/or kits for increasing the sensitivity of cells and/or tumours to ionizing radiation.

As will be discussed in more detail below, there is described PCLX-001 (also known as DDD86481) and uses thereof.

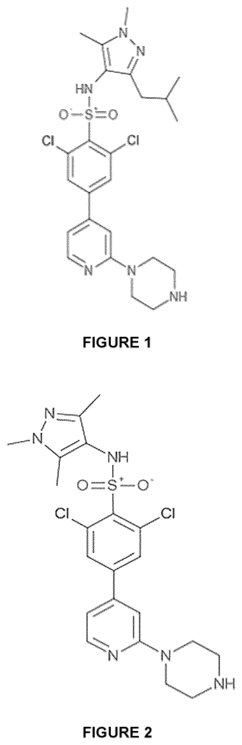

The structure of PCLX-001 is as follows (see FIG. 1).

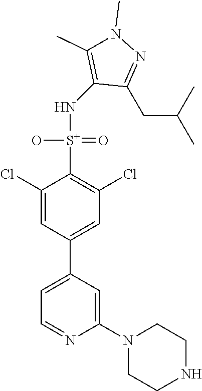 [SMILES: Cc1c(N[S+](=O)(O)c2c(Cl)cc(-c3ccnc(N4CCNCC4)c3)cc2Cl)c(CC(C)C)nn1C]

The structure of DDD85646 (PCLX-002) is as follows (see FIG. 2).

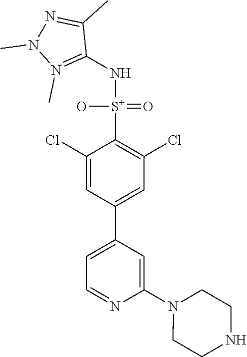

PCLX-001 (also known as DDD86481) and PCLX-002 (also known as DDD85646) are described in WO 2010/026365.

In one aspect, there is provided a radiosensitizer and/or composition including a radiosensitizer.

In a specific example, the radiosensitizer may be PCLX-001 (also known as DDD86481).

In a specific example, the radiosensitizer may be PCLX-002 (also known as DDD85646).

Examples of know radiosensitizers include, but are not limited to nitrosoureas, such carmistine (BCNU) or lomustine (CCNU), platinum-based drugs, and anthracyclines such as doxorubicin, and topoisomerase inhibitors such as etoposide.

The term “radiosensitizer”, as used herein refers to an agent, molecule, compound or composition that enhances the sensitivity of a neoplastic cell, a cancer cell and/or a tumor to the effects of radiation. The “sensitivity” of a neoplastic cell, a cancer cell, and/or a tumour to radiation is the susceptibility of the neoplastic cell, cancer cell, and/or tumour to the inhibitory effects of radiation on the cell's or tumour's growth and/or viability.

In a specific example, the radiosensitizer is a compound and/or composition that may increase sensitivity of a cell and/or tumour to radiation.

In some examples, “radiosensitization” refers to rendering a cell more sensitive to radiation. Radiosensitization of a cell prior to radiation treatment may increase its susceptibility to radiation compared to a cell that has not been radiosensitized prior to radiation treatment.

In some examples, a radiosensitizer may increase the effectiveness of radiation therapy, reduce the side effects of radiation therapy, or improve the overall response to treatment.

In some aspects, there is provided use of a radiosensitizer and a radiation therapy in the treatment of a subject having a cancer, suspected of having a cancer, or at risk of developing a cancer.

Examples of radiation therapy include, but are not limited to, thoracic irradiation, irradiation onto bone metastasis site, irradiation onto lymph node metastasis, irradiation onto adrenal metastasis, irradiation onto liver metastasis, irradiation onto primary brain tumors, irradiation onto primary brain cancers, irradiation onto cancer metastatic to the brain, and the like.

In one example, there may be provided the use of PCLX-001 (also known as DDD86481) and a radiation therapy in the treatment of a subject having a cancer, suspected of having a cancer, or at risk of developing a cancer.

In one example, there may be provided the use of PCLX-002 (also known as DDD85646) and a radiation therapy in the treatment of a subject having a cancer, suspected of having a cancer, or at risk of developing a cancer.

The term “subject”, as used herein, refers to an animal, and can include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal.

In a specific example, the subject is a human.

The term “treatment” or “treat” as used herein, refers to obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment.

The term “prevent” or “prevention” refers to prophylactic or preventative measures that prevent and/or slow the development of a targeted pathologic condition or disorder, such as cancer. Thus, those in need of prevention include those at risk of or susceptible to developing the disorder. In certain embodiments, a disease or disorder is successfully prevented according to the methods provided herein if the patient develops, transiently or permanently, e.g., fewer or less severe symptoms associated with the disease or disorder, or a later onset of symptoms associated with the disease or disorder, than a patient who has not been subject to the methods of the invention.

In some examples, treatment results in prevention or delay of onset or amelioration of symptoms of a disease in a subject or an attainment of a desired biological outcome.

The term “diagnosis” as used herein, refers to the identification of a molecular and/or pathological state, disease or condition, such as the identification of a cancer.

The term “alleviates” as used herein refers to a decrease, reduction or elimination of a condition, disease, disorder, or phenotype, including an abnormality or symptom.

The term “cancer”, as used herein, refers to a variety of conditions caused by the abnormal, uncontrolled growth of cells. Cells capable of causing cancer, referred to as “cancer cells”, possess characteristic properties such as uncontrolled proliferation, immortality, metastatic potential, rapid growth and proliferation rate, and/or certain typical morphological features. Cancer cells may be in the form of a tumour, but such cells may also exist alone within a subject, or may be a non-tumorigenic cancer cell.

A cancer can be detected in any of a number of ways, including, but not limited to, detecting the presence of a tumor or tumors (e.g., by clinical or radiological means), examining cells within a tumor or from another biological sample (e.g., from a tissue biopsy), measuring blood markers indicative of cancer, and detecting a genotype indicative of a cancer. However, a negative result in one or more of the above detection methods does not necessarily indicate the absence of cancer, e.g., a patient who has exhibited a complete response to a cancer treatment may still have a cancer, as evidenced by a subsequent relapse.

It will be appreciated that, in general, determination of the severity of disease requires identification of certain disease characteristics, for example, whether the cancer is pre-metastatic or metastatic, the stage and/or grade of cancer, and the like.

Staging is a process used to describe how advanced a cancer is in a subject. Staging may be important in determining a prognosis, planning treatment and evaluating the results of such treatment. While different cancer staging systems may need to be used for different types of cancer, most staging systems generally involve describing how far the cancer has spread anatomically and attempt to put subjects with similar prognosis and treatment in the same staging group.

Examples of common staging systems used for most solid tumours, some leukemia's and lymphomas are the Overall Stage Grouping system and the TMN system. In the Overall Stage Grouping system, Roman numerals I through IV are utilized to denote the four stages of a cancer. Generally, if a cancer is only detectable in the area of the primary lesion without having spread to any lymph nodes it is called Stage I. Stage II and III cancers are generally locally advanced and/or have spread to the local lymph nodes. For example, if the cancer is locally advanced and has spread only to the closest lymph nodes, it is called Stage II. In Stage III, the cancer is locally advanced and has generally spread to the lymph nodes in near proximity to the site of the primary lesion. Cancers that have metastasized from the primary tumour to a distant part of the body, such as the liver, bone, brain or another site, are called Stage IV, the most advanced stage. Accordingly, stage I cancers are generally small localized cancers that are curable, while stage IV cancers usually represent inoperable or metastatic cancers. As with other staging systems, the prognosis for a given stage and treatment often depends on the type of cancer. For some cancers, classification into four prognostic groups is insufficient and the overall staging is further divided into subgroups. In contrast, some cancers may have fewer than four stage groupings.

A cancer that recurs after all visible tumour has been eradicated is generally called recurrent disease, with local recurrence occurring in the location of the primary tumour and distant recurrence representing distant metastasis.

Variations to the staging systems may depend on the type of cancer. Moreover, certain types of cancers. The staging system for individual cancers maybe revised with new information and subsequently, the resulting stage may change the prognosis and treatment for a specific cancer.

The “grade” of a cancer may be used to describe how closely a tumour resembles normal tissue of its same type. Based on the microscopic appearance of a tumour, pathologists identify the grade of a tumour based on parameters such as cell morphology, cellular organization, and other markers of differentiation. As a general rule, the grade of a tumour corresponds to its rate of growth or aggressiveness and tumours are typically classified from the least aggressive (Grade I) to the most aggressive (Grade IV).

Accordingly, the higher the grade, the more aggressive and faster growing the cancer. Information about tumour grade is useful in planning treatment and predicting prognosis.

In one example, the cancer is a brain cancer.

In some examples, brain cancers include astrocytomas, gliomas, embryonal tumours, non-malignant brain tumors, pediatric brain tumours, or metastatic tumours.

Examples of astrocytomas include, but are not limited to, anaplastic astrocytoma, diffuse astrocytoma, pilocytic astrocytoma, or glioblastoma.

Examples of gliomas include, but are not limited to, diffuse midline glioma, oligodendroglioma, ependymoma, or optic pathway glioma.

Examples of embryonal tumours include, but are not limited to atypical teratoid/rhaboid (AT/RT), or medulloblastoma.

Examples of non-malignant brain tumours include, but are not limited to, acoustic neuroma, meningioma, pituitary adenomas, craniopharyngioma, or pilocytic astrocytoma.

Examples of pediatric brain tumours include, but are not limited to, atypical teratoid/rhaboid tumour (AT/RT), diffuse midline glioma, medulloblastoma, pilocytic astrocytoma, craniopharyngioma, ependymoma, or optic pathway glioma.

Other brain tumour and related conditions include, but are not limited to, brain cysts, dysembryoplastic neuroepithelial tumour (DNET), germ cell tumours, subependymomas, neurofibromatosis type I, pineal tumours, chordoma, ganglioblastoma, neurofibromatosis type 2, or tuberous sclerosis complex (TSC).

Brain cancers may be primary or secondary brain cancer.

In one example, a “primary brain cancer” is an intracranial cancer of central nervous system cells.

A “secondary brain cancer” is a cancer located in the central nervous system that includes cells metastasized from other areas of the body, including but not limited to a solid tumor cancer, breast cancer, lung cancer, colorectal cancer, endometrial cancer, ovarian cancer, testicular cancer, pancreatic cancer, pancreatic ductal adenocarcinoma, cancer of the adrenal cortex, non-Hodgkin's lymphoma, multiple myeloma, leukemia, Kaposi's sarcoma, Ewing's sarcoma, soft tissue sarcoma, nephroblastoma, glioblastoma multiforme, prostate cancer, liver cancer, bone cancer, chondrosarcoma, renal cancer, bladder cancer, and gastric cancer.

In some examples, the brain cancer is primary brain cancer. In some examples, the brain cancer is metastatic brain cancer.

In some examples, in the case of lymphoma, Stage I refers to lymphoma in only one group of lymph nodes. Stage II refers to two or more groups of lymph nodes are affected but they are all either above or below the diaphragm, either all in the chest or all in the abdomen. Stage III refers to two or more groups of lymph nodes are affected in both the chest and the abdomen. Stage IV refers to lymphoma is in at least one organ (e.g., bone marrow, liver or lungs) as well as the lymph nodes. Additional designations may be added to the foregoing stages. For example, “A” generally means the patient has not experiences any troublesome symptoms. “B” means the patient has experienced B symptoms (e.g., fever, night sweats, weight loss). X means the patient has bulky disease (e.g., large tumour greater than 10 cm in size). E means the patient has extranodal disease (e.g., disease outside the lymph nodes).

In some examples, a radiosensitizer may be used in the treatment of breast cancer, head and neck cancer, prostate cancer, pancreatic cancer, or lymphoma.

In some examples, a radiosensitizer may be used in the treatment of anaplastic large cell lymphoma, acute myeloid leukemia, Blast Phase Chronic Myeloid Leukemia, Burkitt's Lymphoma, Plasma Cell Myeloma, Intestinal Adenocarcinoma, Intestinal squamous cell carcinoma, Lung Squamous Carcinoma, Lung Adenocarcinoma, Lung mixed Adenosquamous Carcinoma, Lung Small Cell Carcinoma, Lung, Oesophagus Squamous Cell Carcinoma, Bone, Breast Ductal Carcinoma, Breast Lobular Carcinoma, Stomach Diffuse Adenocarcinoma, Thyroid Medullary Carcinoma, urinary Tract Transitional Cell Carcinoma, myeloma, ovarian clear cell carcinoma, transition cell carcinoma (ureter and bladder cancer), neuroendocrine cancers, chronic myelogenous leukemia (CML), lymphoma-CLL, breast carcinoma, colorectal adenocarcinoma, pancreas adenocarcinoma, ovarian carcinoma, non-small cell lung carcinoma, osteosarcoma, melanoma, gastric adenocarcinoma, endometrial adenocarcinoma, cholangiocarcinoma (bile duct cancer), gallbladder cancer, liver cancer(s), esophageal squamous carcinoma, or primary brain cancers and primary brain tumors.

In some examples, a radiosensitizer may be used in the treatment of brain cancers include astrocytomas, gliomas, gangliomas, gangliogliomas, ependymomas, meningiomas, primary CNS lymphoma, neuroblastoma, embryonal tumours, non-malignant brain tumors, pediatric brain tumours, or metastatic tumours.

In some examples, a radiosensitizer may be used in the treatment of anaplastic astrocytoma, diffuse astrocytoma, pilocytic astrocytoma, xanthoastrocytomas, or glioblastoma.

In some examples, a radiosensitizer may be used in the treatment of diffuse midline glioma, oligodendroglioma, ependymoma, or optic pathway glioma.

In some examples, a radiosensitizer may be used in the treatment of atypical teratoid/rhaboid (AT/RT), or medulloblastoma.

In some examples, a radiosensitizer may be used in the treatment of acoustic neuroma, meningioma, pituitary adenomas, craniopharyngioma, or pilocytic astrocytoma.

In some examples, a radiosensitizer may be used in the treatment of atypical teratoid/rhaboid tumour (AT/RT), diffuse midline glioma, medulloblastoma, pilocytic astrocytoma, craniopharyngioma, ependymoma, or optic pathway glioma.

In some examples, a radiosensitizer may be used in the treatment of brain cysts, dysembryoplastic neuroepithelial tumour (DNET), germ cell tumours, subependymomas, neurofibromatosis type I, pineal tumours, chordoma, ganglioblastoma, neurofibromatosis type 2, or tuberous sclerosis complex (TSC).

In some examples, a radiosensitizer may be used in the treatment of primary or secondary brain cancer.

In some examples, a radiosensitizer may be used in the treatment of intracranial cancer of central nervous system cells.

In some examples, a radiosensitizer may be used in the treatment of a cancer located in the central nervous system that includes cells metastasized from other areas of the body, including but not limited to a solid tumor cancer, breast cancer, lung cancer, colorectal cancer, endometrial cancer, ovarian cancer, testicular cancer, pancreatic cancer, pancreatic ductal adenocarcinoma, cancer of the adrenal cortex, non-Hodgkin's lymphoma, multiple myeloma, leukemia, Kaposi's sarcoma, Ewing's sarcoma, soft tissue sarcoma, nephroblastoma, glioblastoma multiforme, prostate cancer, liver cancer, bone cancer, chondrosarcoma, renal cancer, bladder cancer, and gastric cancer.

In some examples, a radiosensitizer may be used in the treatment of a primary brain cancer. In some examples, the brain cancer is metastatic brain cancer.

In another embodiment, there is provided a method wherein the subject is a child, adolescent, adult, or elderly. In another embodiment, the subject is male or female. In another embodiment, the subject is a human.

The term “lymphoma” generally refers to a malignant neoplasm of the lymphatic system, including cancer of the lymphatic system. The two main types of lymphoma are Hodgkin's disease (HD or HL) and non-Hodgkin's lymphoma (NHL). Abnormal cells appear as congregations which enlarge the lymph nodes, form solid tumours in the body, or more rarely, like leukemia, circulate in the blood. Hodgkin's disease lymphomas, include nodular lymphocyte predominance Hodgkin's lymphoma; classical Hodgkin's lymphoma; nodular sclerosis Hodgkin's lymphoma; lymphocyte rich classical Hodgkin's lymphoma; mixed cellularity Hodgkin's lymphoma; lymphocyte depletion Hodgkin's lymphoma. Non-Hodgkin's lymphomas include small lymphocytic NHL, follicular NHL; mantle cell NHL; mucosa-associated lymphoid tissue (MALT) NHL; diffuse large cell B-cell NHL; mediastinal large B-cell NHL; precursor T lymphoblastic NHL; cutaneous T-cell NHL; T-cell and natural killer cell NHL; mature (peripheral) T-cell NHL; Burkitt's lymphoma; mycosis fungoides; Sezary Syndrome; precursor B-lymophoblastic lymphoma; B-cell small lymphocytic lymphoma; lymphoplasmacytic lymphoma; splenic marginal zone B-cell lymphoma; nodal marginal zone lymphoma; plasma cell myeloma/plasmacytoma; intravascular large B-cell NHL; primary effusion lymphoma; blastic natural killer cell lymphoma; enteropathy-type T-cell lymphoma; hepatosplenic gamma-delta T-cell lymphoma; subcutaneous panniculitis-like T-cell lymphoma; angioimmunoblastic T-cell lymphoma; and primary systemic anaplastic large T/null cell lymphoma.

In some examples, the compounds and/or compositions described herein (for example, PCLX-001 or PCLX-002) may be used as a radiosensitizer to treat various stages and grades of cancer development and progression.

In some examples, PCLX-001 or PCLX-002 may be used as a radiosensitizer in the treatment of early stage cancers including early neoplasias that may be small, slow growing, localized and/or nonaggressive, for example, with the intent of curing the disease or causing regression of the cancer, as well as in the treatment of intermediate stage and in the treatment of late stage cancers including advanced and/or metastatic and/or aggressive and/or recurrent neoplasias, for example, to slow the progression of the disease, to reduce metastasis or to increase the survival of the patient.

Similarly, PCLX-001 or PCLX-002 may be used as a radiosensitizer in the treatment of low grade cancers, intermediate grade cancers and or high grade cancers.

In some examples, it is contemplated that PCLX-001 or PCLX-002 may be used as a radiosensitizer in the treatment of indolent cancers, recurrent cancers including locally recurrent, distantly recurrent and/or refractory cancers (i.e., cancers that have not responded to treatment), metastatic cancers, locally advanced cancers and aggressive cancers

The term “inhibit” or “inhibitor” as used herein, refers to any method or technique which inhibits protein synthesis, levels, activity, or function, as well as methods of inhibiting the induction or stimulation of synthesis, levels, activity, or function of the protein of interest. In some example, the term also refers to any metabolic or regulatory pathway, which can regulate the synthesis, levels, activity, or function of the protein of interest. The term includes binding with other molecules and complex formation. Therefore, the term “inhibitor” refers to an agent or compound, the application of which results in the inhibition of protein function or protein pathway function. However, the term does not imply that each and every one of these functions must be inhibited at the same time.

As used herein, “radiation therapy” refers to the use of high-energy radiation to treat a subject, including but not limited to, external radiation therapy and internal radiation therapy, which may also be referred to as brachytherapy.

In some examples, external radiation therapy involves directing a beam of direct or indirect ionizing radiation to a tumor or cancer site. The beams of radiation, the photons, the Cobalt or the particle therapy may be focused to the tumor or cancer site. It will be appreciated that using this therapy it will be is nearly impossible to avoid exposure of normal, healthy tissue.

External radiation therapy may be used to treat most types of cancer, including but not limited to, cancer of the bladder, brain, breast, cervix, larynx, lung, prostate, and vagina. Intraoperative radiation therapy (IORT) is a form of external radiation that is given during surgery, and can be used to treat localized cancers that cannot be completely removed or that have a high risk of recurring in nearby tissues, including, but not limited to treatment of thyroid and colorectal cancers, gynecological cancers, cancer of the small intestine, and cancer of the pancreas. Prophylactic cranial irradiation (PCI) is another type of external radiation given to the brain when the primary cancer (for example, small cell lung cancer) has a high risk of spreading to the brain.

Energy sources for external radiation therapy include, but are not limited to, direct or indirect ionizing radiation, for example: x-rays, gamma rays and particle beams or combination thereof.

In some examples, internal radiation therapy, also referred to as brachytherapy, involves implanting a radiation-emitting source, such as beads, wires, pellets, capsules, etc., inside the body, at, or near to the tumor site. Energy source for internal radiation therapy may be radioactive isotopes comprising: iodine (iodine-125 or iodine-131), strontium-89, radioisotopes of phosphorous, palladium, cesium, indium, phosphate, or cobalt, and combination thereof. Such implants can be removed following treatment, or left in the body inactive. Types of internal radiation therapy include, but are not limited to, interstitial, and intracavity brachytherapy (high dose rate, low dose rate, pulsed dose rate). Internal radiation therapy may also include biological carriers of radioisotopes, such as with radio-immunotherapy wherein tumor-specific antibodies bound to radioactive material are administered to a patient. The antibodies bind tumor antigens, thereby effectively administering a dose of radiation to the relevant tissue.

In a specific example, the radiation is γ-radiation. In one example, the ionizing radiation is X-rays generated by a linear accelerator (Linac).

In other examples, radiation therapy that may be used in combination with CER therapy include external beam radiation therapy (e.g., conventional external beam radiation therapy, stereotactic radiation, 3-dimensional conformal radiation therapy, intensity-modulated radiation therapy, volumetric modulated arc therapy, particle therapy, proton therapy, and auger therapy), brachytherapy, systemic radioisotope therapy, intraoperative radiotherapy, or any combination thereof.

Methods of administering radiation therapy are well known to those of skill in the art.

The compound or compositions of the radiosensitizer(s) described may be formulated for and used in any route of administration such as, for example, intranasal administration; oral administration; inhalation administration; subcutaneous administration; transdermal administration; intra-arterial administration, with or without occlusion; intracranial administration; intraventricular administration; intravenous administration; buccal administration; intraperitoneal administration; intraocular administration; intramuscular administration; implantation administration; and central venous administration.

In some examples there is provided intracranial administration of a radiosensitizer to a selected target tissue, including injection of an aqueous solution of a radiosensitizer and implantation of a controlled release system, such as a targeted delivery radiosensitizer incorporating polymeric implant at the selected target site. Use of a controlled release implant reduces the need for repeat injections. Intracranial implants are known.

In some examples, multiple administrations or courses of therapy with respect to radiotherapy, a radiosensitizer compound or a composition comprising a radiosensitizer compound, or both may be given to the subject.

In some examples, a patient may be exposed to at least one course of radiotherapy within, within at least, or within at most 5, 10, 15, 20, 25, 30, 35, 40, 45, 50, 55 minutes or 1, 2, 3, 10, 11, 12, 13, 14, 15, 16, 17, 18, 19, 20, 21, 22, 23, 24, 30, 36, 42, 48, 54, 86, 84, 96, 96, 102, 108, 114, 120, 126, 130, 136, 142 hours, or 1, 2, 3, 4, 5, 6, 7 days, or 1, 2, 3, 4, 5, 6, 7, 8, 9, 10, 11, 12 weeks or more, or a combination thereof, of the time that the patient is a radiosensitizer compound or a composition comprising a radiosensitizer compound.

In some examples, a pharmaceutically effective amount of PCLX-001 is used. In some examples, a therapeutically effective amount of PCLX-001 is used.

The term “pharmaceutically effective amount” or “effective amount” as used herein refers to the amount of a drug or pharmaceutical agent, such as PCLX-001, that will elicit the biological or medical response of a tissue, system, animal or human that is being sought by a researcher or clinician. This amount can be a “therapeutically effective amount”. These terms refer to the amount of a compound and/or compositions described herein which treats, upon single or multiple dose administration, a subject with a disease or condition. An effective amount can be readily determined by the attending diagnostician, as one skilled in the art, by the use of known techniques and by observing results obtained under analogous circumstances. In determining the effective amount, the dose, a number of factors are considered by the attending diagnostician, including, but not limited to: the species of the subject; its size, age, and general health; the specific condition, disorder, or disease involved; the degree of or involvement or the severity of the condition, disorder, or disease, the response of the individual subject; the particular compound administered; the mode of administration; the bioavailability characteristics of the preparation administered; the dose regimen selected; the use of concomitant medication; and other relevant circumstances.

Thus, the term “therapeutically effective amount”, as used herein, refers to an amount effective, at dosages and for periods of time necessary to achieve the desired result. Effective amounts may vary according to factors such as the disease state, age, sex and/or weight of the subject. The amount of a given compound or composition that will correspond to such an amount will vary depending upon various factors, such as the given drug or compound, the pharmaceutical formulation, the route of administration, the identity of the subject being treated, and the like, but can nevertheless be routinely determined by one skilled in the art.

The term “pharmaceutically acceptable” as used herein includes compounds, materials, compositions, and/or dosage forms (such as unit dosages) which are suitable for use in contact with the tissues of a subject without excessive toxicity, irritation, allergic response, or other problem or complication, commensurate with a reasonable benefit/risk ratio. Each carrier, excipient, etc. is also “acceptable” in the sense of being compatible with the other ingredients of the formulation.

The term “excipient” means a pharmacologically inactive component such as a diluent, lubricant, surfactant, carrier, or the like. Excipients that are useful in preparing a pharmaceutical composition are generally safe, non-toxic and are acceptable for human pharmaceutical use. Reference to an excipient includes both one and more than one such excipient.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintegrants (e.g., potato starch or sodium starch glycolate), stabilizers and preservatives, and the like.

The pharmaceutical compositions may be in a form suitable for oral use, for example, as tablets, troches, lozenges, aqueous or oily suspensions, dispersible powders or granules, emulsion hard or soft capsules, or syrups or elixirs. Compositions intended for oral use may be prepared according to methods known to the art for the manufacture of pharmaceutical compositions and may contain one or more agents selected from the group of sweetening agents, flavouring agents, colouring agents and preserving agents in order to provide pharmaceutically elegant and palatable preparations. Tablets contain die active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

Pharmaceutical compositions for oral use may also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Aqueous suspensions contain the active compound in admixture with suitable excipients including, for example, suspending agents, such as sodium carboxymethylcellulose, methyl cellulose, hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, one or more colouring agents, one or more flavouring agents or one or more sweetening agents, such as sucrose or saccharin.

Oily suspensions may be formulated by suspending the active ingredients in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may be added to provide palatable oral preparations. These compositions can be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Pharmaceutical compositions of the invention may also be in the form of oil-in-water emulsions. The oil phase maybe a vegetable oil, for example, olive oil or arachis oil, or a mineral oil, for example, liquid paraffin, or it may be a mixtures of these oils.

Suitable emulsifying agents maybe naturally-occurring gums, for example, gum acacia or gum tragacanth; naturally-occurring phosphatides, for example, soybean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions may also contain sweetening and flavouring agents.

Syrups and elixirs may be formulated with sweetening agents, for example, glycerol, propylene glycol, sorbitol or sucrose. Such formulations may also contain a demulcent, a preservative, and/or flavouring and colouring agents.

The pharmaceutical compositions may be in the form of a sterile injectable aqueous suspension. This suspension may be formulated according to known art using suitable dispersing or wetting agents, and suspending agents such as those mentioned above. The sterile injectable preparation may also be sterile injectable solution or suspension in a non-toxic parentally acceptable diluent or solvent, for example, as a solution in 1,3-butanediol. Acceptable vehicles and solvents that may be employed include, but are not limited to, water, Ringer's solution, lactated Ringer's solution and isotonic sodium chloride solution. Other examples are, sterile, fixed oils which are conventionally employed as a solvent or suspending medium, and a variety of bland fixed oils including, for example, synthetic mono- or diglycerides. In addition, fatty acids such as oleic acid find use in the preparation of injectables.

The compounds and compositions may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intratumoral, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot/for example, subcutaneously or intramuscularly.

As used herein, the terms “contacting” refers to a process by which, for example, a compound may be delivered to a cell. The compound may be administered in a number of ways, including, but not limited to, direct introduction into a cell (i.e., intracellularly) and/or extracellular introduction into a cavity, interstitial space, or into the circulation of the organism.

Thus, in some example, contacting occurs in vivo. In other examples, contacting may occur in vitro.

A “cell” refers to an individual cell or cell culture. In one example, the cell is a cell obtained or derived from a subject. The culturing of cells and suitable culture media are known.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection.

The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

The compounds and/or compositions described herein may be administered either simultaneously (or substantially simultaneously) or sequentially, dependent upon the condition to be treated, and may be administered in combination with other treatment(s). The other treatment(s), may be administered either simultaneously (or substantially simultaneously) or sequentially.

A “treatment or dosage regimen” as used herein refers to a combination of dosage, frequency of administration, or duration of treatment, with or without addition of a second medication.

A compound or composition may be administered alone or in combination with other treatments, either simultaneously or sequentially, dependent upon the condition to be treated.

In treating a subject, a therapeutically effective amount may be administered to the subject.

In some examples, therapeutic formulations comprising the compounds or compositions as described herein may be prepared for by mixing compounds or compositions having the desired degree of purity with optional physiologically acceptable carriers, excipients or stabilizers, in the form of aqueous solutions, lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as Tween™, Pluronics™ or polyethylene glycol (PEG).

The therapeutic formulation may also contain more than one active compound as necessary for the particular indication being treated, typically those with complementary activities that do not adversely affect each other. Such molecules are suitably present in combination in amounts that are effective for the purpose intended.

For example, known treatments are dependent upon the subject being treated, the type of disease, and its stage.

Existing treatment modalities for various cancers, are known to the skilled worker.

Accordingly, known treatments for cancer(s) may be used together with PCLX-001.

Common drugs used in treating primary and secondary brain cancer include, but are not limited to conjugates of methotrexate, vinca alkaloids, carmustine, cisplatin, carboplatin, nitrosourea, hydroxyurea, cyclophosphamide, etoposide, procarbazine, irinotecan, lomustine, vincristine, bevacizumab, vorasidenib, ivosidenib, tucatinib, capecitabine, trastuzumab, and/or temozolomide.

In the case of treatment of a subject with dexamethasone, ulcers may develop in the subject. Accordingly, in some examples, a subject being treated with dexamethasone may also be treated with a proton pump inhibitor.

As used herein, the term “proton pump inhibitor” refers to a class of compounds that reduce or down-regulate the production of stomach acid. Typically, a PPI functions by inhibiting the hydrogen/potassium adenosine triphosphatase (H+/K+ATPase) enzyme system in the stomach.

Protein pump inhibitors include but are not limited to Omeprazole, Lansoprazole, Dexlansoprazole, Esomeprazole, Pantoprazole, Rabeprazole and Ilaprazole.

In some examples, PPI's may reduce PCLX-001 absorption by reducing gastric acidity.

Common drug combinations for use in treating lymphomas include, but are not limited, to CHOP (i.e., cyclophosphamide, doxorubicin, vincristine, and prednisone), GAP-BOP (i.e., cyclophosphamide, doxorubicin, procarbazine, bleomycin, vincristine, and prednisone), m-BACOD (i.e., methotrexate, bleomycin, doxorubicin, cyclophosphamide, vincristine, dexamethasone, and leucovorin), ProMACE-MOPP (i.e., prednisone, methotrexate, doxorubicin, cyclophosphamide, etoposide, leucovorin with standard MOPP), ProMACE-CytaBOM (prednisone, doxorubicin, cyclophosphamide, etoposide, cytarabine, bleomycin, vincristine, methotrexate, and leucovorin), and MACOP-B (methotrexate, doxorubicin, cyclophosphamide, vincristine, prednisone, bleomycin, and leucovorin). For relapsed aggressive non-Hodgkin's lymphoma the following chemotherapy drug combinations may be used with the compounds and compositions described herein: IMVP-16 (i.e., ifosfamide, methotrexate, and etoposide), MIME (i.e., methyl-gag, ifosfamide, methotrexate, and etoposide), DHAP (i.e., dexamethasone,—16 high dose cytarabine, and cisplatin), ESHAP (i. e., etoposide, methylprednisone, high dosage cytarabine, and cisplatin), CEFF(B) (i.e., cyclophosphamide, etoposide, procarbazine, prednisone, and bleomyxacin), and CAMP (i.e., lomustine, mitoxantrone, cytarabine, and prednisone).

Treatment for salvage chemotherapy used for certain lymphomas such as for relapsed, resistant Hodgkin's Disease include but are not limited to VABCD (i.e., vinblastine, doxorubicin, dacarbazine, lomustine and bleomycin), ABDIC (i.e., doxorubicin, bleomycin, dacarbazine, lomustine, and prednisone), CBVD (i.e., lomustine, bleomycin, vinblastine, dexamethasone), PCVP (i.e., vinblastine, procarbazine, cyclophosphamide, and prednisone), CEP (i.e., lomustine, etoposide, and prednimustine), EVA (i.e., etoposide, vinblastine, and doxorubicin), MOPLACE (i.e., cyclophosphamide, etoposide, prednisone, methotrexate, cytaravine, and vincristine), MIME (i.e., methyl-gag, ifosfamide, methotrexate, and etoposide), MINE (i.e., mitoquazone, ifosfamide, vinorelbine, and etoposide), MTX-CHOP (i.e., methotrexate and CHOP), CEM (i.e., lomustine, etoposide, and methotrexate), CEVD (i.e., lomustine, etoposide, vindesine, and dexamethasone), CAVP (i.e., lomustine, melphalan, etoposide, and prednisone), EVAP (i.e., etoposide, vinblastine, cytarabine, and cisplatin), and EPOCH (i.e., etoposide, vincristine, doxorubicin, cyclophosphamide, and prednisone).

Treatment for breast cancer includes treatment with paclitaxel, docetaxel, nanoparticle albumin bound paclitaxel, capecitabine, doxorubicin, eribulin, vinorelbine, trastuzumab, tucatinib, carboplatin, cisplatin; endocrine therapies including tamoxifen, anastrozole, letrozole, exemestane, fulvestrant, and cell cycle inhibitors including palbocilib, ribociclib, LEE001, and everolimus; immune checkpoint inhibiting drugs including nivolumab and pembrolizumab, either individually or in combination.

Treatment for small cell lung cancer includes treatment with carboplatin, cisplatin, etoposide, irinotecan, atezolizumab, and pembrolizumab, either individually or in combination.

Treatment for non-small cell lung cancer includes treatment with carboplatin, paclitaxel, docetaxel, gefitinib, erlotinib, afatinib, bevacizumab, ramucirumab, osimertinib, necitumumab, crizotinib, ceritinib, alectinib, and immune checkpoint inhibiting drugs including nivolumab, pembrolizumab, either individually or in combination.

Treatment for bladder carcinoma includes treatment with methotrexate, vinblastine, doxorubicin, cisplatin; carboplatin and paclitaxel; docetaxel, gemcitabine and cisplatin, either individually or in combination.

A skilled worked will be able to determine the appropriate dose for the individual subject by following the instructions on the label. Preparation and dosing schedules for commercially available second therapeutic and other compounds administered in combination with or concomitantly with compounds or compositions described herein may be used according to manufacturers' instructions or determined empirically by the skilled practitioner.

Factors which may be taken into account when determining an appropriate dosage include the severity of the disease state, general health of the subject, age, weight, and gender of the subject, diet, time and frequency of administration, the particular components of the combination, reaction sensitivities, and tolerance/response to therapy.

As described herein, there is also provided the following embodiments:
1. Use of PCLX-001, or pharmaceutically acceptable salt thereof, for radiosensitizing a cancerous cell in a subject in need of radiation therapy.
2. Use of PCLX-001, or pharmaceutically acceptable salt thereof, in the manufacture of a medicament for radiosensitizing a cancerous cell in a subject in need of radiation therapy.
3. The use of embodiment 1 or 2, wherein said radiation therapy is external radiation therapy, internal radiation therapy or systemic radiation therapy.
4. The use of any one of embodiments 1 to 3, wherein said subject has cancer of the bladder, brain, breast, cervix, larynx, lung, prostate, vagina, thyroid, pancreas, ovary, breast, uterus, gallbladder, perianal and pelvic regions; colorectal cancers; gynecological cancers; cancer of the small intestine; small cell lung cancer; head and neck cancer; bronchial cancer; oral cancer; rectal cancer; tracheal cancer; or adult non-Hodgkin lymphoma.
5. The use of embodiment 4, wherein the brain cancer is an astrocytoma, glioma, embryonal tumour, non-malignant brain tumor, pediatric brain tumour, or metastatic tumours.
6. The use of embodiment 5, wherein the astrocytoma is anaplastic astrocytoma, diffuse astrocytoma, pilocytic astrocytoma, or glioblastoma.
7. The use of embodiment 5, wherein the glioma is diffuse midline glioma, oligodendroglioma, ependymoma, or optic pathway glioma.
8. The use of embodiment 5, wherein the embryonal tumour is atypical teratoid/rhaboid (AT/RT), or medulloblastoma.
9. The use of embodiment 5, wherein the non-malignant brain tumour is acoustic neuroma, meningioma, pituitary adenomas, craniopharyngioma, or pilocytic astrocytoma.
10. The use of embodiment 5, wherein the pediatric brain tumour is atypical teratoid/rhaboid tumour (AT/RT), diffuse midline glioma, medulloblastoma, pilocytic astrocytoma, craniopharyngioma, ependymoma, or optic pathway glioma.
11. The use of any one of embodiments 1 to 10, wherein said subject is a human.
12. The use of any one of embodiments 1 to 11, wherein the compound PCLX-001 or pharmaceutiall acceptable salt thereof is at a dosing range of about 50 nM to about 150 nM.
13. Use of a radiosensitizer, wherein the radiosensitizer is PCLX-001, and use of radiation therapy, for treating a subject with cancer, suspected of having a cancer, or at risk of developing a cancer.
14. Use of a radiosensitizer, wherein the radiosensitizer is PCLX-001, in the manufacture of a medicament, and and use of radiation therapy, for treating a subject with cancer, suspected of having a cancer, or at risk of developing a cancer.
15. The use of embodiment 13, or 14 wherein said radiation therapy is external radiation therapy, internal radiation therapy or systemic radiation therapy.
16. The use of any one of embodiments 13 to 15, wherein the said subject has cancer of the bladder, brain, breast, cervix, larynx, lung, prostate, vagina, thyroid, pancreas, ovary, breast, uterus, gallbladder, perianal and pelvic regions; colorectal cancers; gynecological cancers; cancer of the small intestine; small cell lung cancer; head and neck cancer; bronchial cancer; oral cancer; rectal cancer; tracheal cancer; or adult non-Hodgkin lymphoma.
17. The use of embodiment 16, wherein the brain cancer is an astrocytoma, glioma, embryonal tumour, non-malignant brain tumor, pediatric brain tumour, or metastatic tumours.
18. The use of embodiment 17, wherein the astrocytoma is anaplastic astrocytoma, diffuse astrocytoma, pilocytic astrocytoma, or glioblastoma.
19. The use of embodiment 17, wherein the glioma is diffuse midline glioma, oligodendroglioma, ependymoma, or optic pathway glioma.
20. The use of embodiment 17, wherein the embryonal tumour is atypical teratoid/rhaboid (AT/RT), or medulloblastoma.
21. The use of embodiment 17, wherein the non-malignant brain tumour is acoustic neuroma, meningioma, pituitary adenomas, craniopharyngioma, or pilocytic astrocytoma.
22. The use of embodiment 17, wherein the pediatric brain tumour is atypical teratoid/rhaboid tumour (AT/RT), diffuse midline glioma, medulloblastoma, pilocytic astrocytoma, craniopharyngioma, ependymoma, or optic pathway glioma.
23. The use of any one of embodiments 13 to 22, further comprising use or one or more drugs for treating brain cancer comprising: conjugates of methotrexate, vinca alkaloids, carmustine, cisplatin, carboplatin, nitrosourea, hydroxyurea, cyclophosphamide, etoposide, procarbazine, irinotecan, lomustine, vincristine, and/or temozolomide.
24. The use of any one of embodiments 13 to 22, further comprising use of one or more drugs in the manufacture of a medicament for treating brain cancer comprising: conjugates of methotrexate, vinca alkaloids, carmustine, cisplatin, carboplatin, nitrosourea, hydroxyurea, cyclophosphamide, etoposide, procarbazine, irinotecan, lomustine, vincristine, and/or temozolomide.
25. The use of any one of embodiments 13 to 24, wherein said subject is a human.
26. The use of any one of embodiments 13 to 23, comprising use of the radiosensitizer at a dosing to achieve blood or target organ or tumor drug concentrations in the range of about 50 nM to about 150 nM.
27. The use of any one of embodiments 13 to 26, comprising use of the radiosensitizer at Time A and use of radiation therapy at Time B for achieving a target dosing range in blood or target organ or tumor drug concentration of about 50 nM to about 150 nM at the cancer site, or at the potential cancer site.
28. The use of embodiment 27, wherein Time B is about 2 hours to about 4 hours after Time A when administering the radiosensitizer at Time A comprises orally administering the radiosensitizer.
29. The use of embodiment 27, wherein Time B is about 72 hours after Time A when administering the radiosensitizer at Time A comprises commencing radiosensitizer therapy.
30. The use of any one of embodiments 1 to 29, wherein administering PCLX-001 comprises PCLX-001 crossing the blood brain barrier.
31. The use of embodiment 30, wherein use of PCLX-001 comprises PCLX-001 crossing the blood brain barrier and acting as a radiosensitizer for primary brain tumors.
32. The use of embodiment 30 or 31, wherein use of PCLX-001 comprises PCLX-001 crossing the blood brain barrier and acting as a radiosensitizer for primary brain tumors about 4 hours after administration.
33. The use of any one of embodiments 30 to 32, wherein use of PCLX-001 comprises PCLX-001 crossing the blood brain barrier and acting as a radiosensitizer for secondary brain tumors.
34. The use of any one of embodiments 30 to 33, wherein use of PCLX-001 comprises PCLX-001 crossing the blood brain barrier and acting as a radiosensitizer for secondary brain tumors about 4 hours after administration.
35. The use of any one of embodiments 13 to 34, wherein the patient discontinues use of a proton pump inhibitor prior to treatment with the radiosensitizer and radiation therapy, if the patient is using the proton pump inhibitor.
36. A method of radiosensitizing a cancerous cell in a subject in need of radiation therapy, comprising: administering to a subject compound PCLX-001, or pharmaceutically acceptable salt thereof.
37. The method of embodiment 36, wherein said radiation therapy is external radiation therapy, internal radiation therapy or systemic radiation therapy.
38. The method of embodiment 36 or 37, wherein said subject has cancer of the bladder, brain, breast, cervix, larynx, lung, prostate, vagina, thyroid, pancreas, ovary, breast, uterus, gallbladder, perianal and pelvic regions; colorectal cancers; gynecological cancers; cancer of the small intestine; small cell lung cancer; head and neck cancer; bronchial cancer; oral cancer; rectal cancer; tracheal cancer; or adult non-Hodgkin lymphoma.
39. The method of embodiment 38, wherein the brain cancer is an astrocytoma, glioma, embryonal tumour, non-malignant brain tumor, pediatric brain tumour, or metastatic tumours.
40. The method of embodiment 39, wherein the astrocytoma is anaplastic astrocytoma, diffuse astrocytoma, pilocytic astrocytoma, or glioblastoma.
41. The method of embodiment 39, wherein the glioma is diffuse midline glioma, oligodendroglioma, ependymoma, or optic pathway glioma.
42. The method of embodiment 39, wherein the embryonal tumour is atypical teratoid/rhaboid (AT/RT), or medulloblastoma.
43. The method of embodiment 39, wherein the non-malignant brain tumour is acoustic neuroma, meningioma, pituitary adenomas, craniopharyngioma, or pilocytic astrocytoma.
44. The method of embodiment 39, wherein the pediatric brain tumour is atypical teratoid/rhaboid tumour (AT/RT), diffuse midline glioma, medulloblastoma, pilocytic astrocytoma, craniopharyngioma, ependymoma, or optic pathway glioma.
45. The method of any one of embodiments 36 to 44, wherein said subject is a human.
46. The method of any one of embodiments 36 to 45, comprising administering the compound PCLX-001 at a dosing range of about 50 nM to about 150 nM.
47. A method of treating a subject with cancer, suspected of having a cancer, or at risk of developing a cancer, comprising administering a radiosensitizer, wherein the radiosensitizer is PCLX-001, and administering radiation therapy.
48. The method of embodiment 47, wherein said radiation therapy is external radiation therapy, internal radiation therapy or systemic radiation therapy.
49. The method of embodiment 47 or 48, wherein the said subject has cancer of the bladder, brain, breast, cervix, larynx, lung, prostate, vagina, thyroid, pancreas, ovary, breast, uterus, gallbladder, perianal and pelvic regions; colorectal cancers; gynecological cancers; cancer of the small intestine; small cell lung cancer; head and neck cancer; bronchial cancer; oral cancer; rectal cancer; tracheal cancer; or adult non-Hodgkin lymphoma.
50. The method of embodiment 49, wherein the brain cancer is an astrocytoma, glioma, embryonal tumour, non-malignant brain tumor, pediatric brain tumour, or metastatic tumours.
51. The method of embodiment 50, wherein the astrocytoma is anaplastic astrocytoma, diffuse astrocytoma, pilocytic astrocytoma, or glioblastoma.
52. The method of embodiment 50, wherein the glioma is diffuse midline glioma, oligodendroglioma, ependymoma, or optic pathway glioma.
53. The method of embodiment 50, wherein the embryonal tumour is atypical teratoid/rhaboid (AT/RT), or medulloblastoma.
54. The method of embodiment 50, wherein the non-malignant brain tumour is acoustic neuroma, meningioma, pituitary adenomas, craniopharyngioma, or pilocytic astrocytoma.
55. The method of embodiment 50, wherein the pediatric brain tumour is atypical teratoid/rhaboid tumour (AT/RT), diffuse midline glioma, medulloblastoma, pilocytic astrocytoma, craniopharyngioma, ependymoma, or optic pathway glioma.
56. The method of any one of embodiments 48 to 55, further comprising administering one or more drugs for treating brain cancer comprising: conjugates of methotrexate, vinca alkaloids, carmustine, cisplatin, carboplatin, nitrosourea, hydroxyurea, cyclophosphamide, etoposide, procarbazine, irinotecan, lomustine, vincristine, and/or temozolomide.
57. The method of any one of embodiments 48 to 56, wherein said subject is a human.
58. The method of any one of embodiments 48 to 57, comprising administering the radiosensitizer at a dosing to achieve blood or target organ or tumor drug concentrations in the range of about 50 nM to about 150 nM.
59. The method of any one of embodiments 48 to 58, comprising administering the radiosensitizer at Time A and administering radiation therapy at Time B for achieving a target dosing range in blood or target organ or tumor drug concentration of about 50 nM to about 150 nM at the cancer site, or at the potential cancer site.
60. The method of embodiment 59, wherein Time B is about 2 hours to about 4 hours after Time A when administering the radiosensitizer at Time A comprises orally administering the radiosensitizer.
61. The method of embodiment 59, wherein Time B is about 72 hours after Time A when administering the radiosensitizer at Time A comprises commencing radiosensitizer therapy.
62. The method of any one of embodiments 36 to 61, wherein administering PCLX-001 comprises PCLX-001 crossing the blood brain barrier.
63. The method of embodiment 62, wherein administering PCLX-001 comprises PCLX-001 crossing the blood brain barrier and acting as a radiosensitizer for primary brain tumors.
64. The method of embodiment 62 or 63, wherein administering PCLX-001 comprises PCLX-001 crossing the blood brain barrier and acting as a radiosensitizer for primary brain tumors about 4 hours after administration.
65. The method of any one of embodiments 62 to 64, wherein administering PCLX-001 comprises PCLX-001 crossing the blood brain barrier and acting as a radiosensitizer for secondary brain tumors.
66. The method of any one of embodiments 26 to 65, wherein administering PCLX-001 comprises PCLX-001 crossing the blood brain barrier and acting as a radiosensitizer for secondary brain tumors about 4 hours after administration.
67. The method of any one of embodiments 47 to 66, wherein the patient discontinues use of a proton pump inhibitor prior to treatment with the radiosensitizer and radiation therapy, if the patient is using the proton pump inhibitor.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES

In general, experiments will be conducted and will include but are not limited to: cell viability studies on various cancer cell lines in irradiation conditions ranging from 2 Gy to 10 Gy with different drug dosing levels to find optimum sensitization effect. Drug dosing is expected to be in range of 10 nM to 500 nM. Radiation and drug exposure would be done concurrently over successive days.

### Example 1—Establishing Drug Cytotoxicity in Dose and Scheduling Schemes Appropriate for Use of PCLX-001 as a Radiosensitizer in Multiple Cell Lines In-Vitro

The effect of PCLX-001 on cellular survival following exposure to PCLX-001 will be measured by clonogenic assays.

Cells will be seeded on 60-mm tissue culture plates at various concentrations to give between about 100-1000 colonies per plate and returned to the incubator overnight to allow the cells to attach. Cells will be incubated with or without PCLX-001 for a period of time. The time period may be up to 24 hours, may be at least 24 hours, or may be up to 96 hours prior to irradiation to allow the drop in myrstolated protein levels in the cancer cells to coincide with the cell's need for these proteins in repairing radiation-related damage. After incubation with PCLX-001, cells will be incubated for a further time period. Colonies will be stained with crystal violet after 10 to 14 days and counted with an automated counting.

Other methods of measuring cytotoxicity are well-known to the skilled person and comprise MTT or MTS assays, Alamar Blue assays, ATP-based assays including bioluminescent assays, the sulforhodamine B (SRB) assay, WST assay, clonogenic assay and the ECIS technology, and may be used.

### Example 2—Radiation and Drug Combination Trials In Vitro Across Multiple Cell Lines, to Access Optimal Timing and of Drug and Radiation Administration Will be Empirically Determined Based on these Studies

The effect of PCLX-001 on cellular survival following exposure to PCLX-001 and ionizing radiation will be measured by clonogenic assays.

Cells will seeded on 60-mm tissue culture plates at various concentrations to give between about 100-1000 colonies per plate and returned to the incubator overnight to allow the cells to attach. For radiosensitization studies, the cells will be incubated with or without PCLX-001 for a period of time before irradiation and then exposed to increasing doses of γ-radiation. After irradiation, cells will be incubated for a further time period. Colonies will be stained with crystal violet after 10 to 14 days and counted with an automated counting.

Other methods of measuring cytotoxicity are well-known to the skilled person and comprise MTT or MTS assays, Alamar Blue assays, ATP-based assays including bioluminescent assays, the sulforhodamine B (SRB) assay, WST assay, clonogenic assay and the ECIS technology, and may be used.

### Example 3—Mechanism of Potential Radiosensitization by Measuring the Most Likely Candidate Pathways of Synergism: PARP-1 Expression, γH2AX Level to Indicate Conversion of Unrepaired Single-Strand DNA Break to Double-Strand DNA Breaks and PI3K Lipid Kinase Activity to Modulate Transformation to More Aggressive Phenotype

In some examples, cell will be treated with PCLX-001 and accessed for PARP-1 and/or γH2AX levels.

In one example, radiolabeled PARP-1 inhibitors will be used in measuring PARP-1 expression and imaging PARP-1 distribution in vivo with PET.

In one example, γH2AX protein level will be measured post-irradiation in the presence and absence of PCLX-001.

In one example, PI3K activity in the presence and absence of PCLX-001 during radiotherapy will be accessed as a marker of cancer cell aggressiveness and oncogenic driving pathways.

### Example 4. Investigate Methods of Cell Death Induced in a Radiosensitization Role by Measuring Apoptotic Fraction of Cell Death. We Will Conclude Whether Apoptosis, or Some Other Cell Death Pathway, Relates to the Effect

Methods of measuring apoptosis are known to a skilled artisan and include, but are not limited to, FACS analysis using Annexin V staining, DNA electrophoresis, uptake of propidium iodide (PI), TUNEL assay, flow cytometry, microscopy, trypan blue or 7AAD.

In some examples, cells may be incubated with anti-Fas antibody to stimulate apoptosis through the extrinsic pathway and 2.5 μM staurosporine (STS) may be used to stimulate apoptosis through the intrinsic pathway. Cycloheximide may be used to inhibit protein translation and to further promote cell death.

In other examples, Western blotting or immunofluorescence, for example probing with specific antibodies that recognize proteins associated with apoptosis, Such as caspases or Bcl-2 family of proteins.

### Example 5—Replicate In Vitro Data In Vivo with Orthotopic and/or Subcutaneous Tumor Models in Mice Treated Orally or by SQ Injection with PCLX-001 in Combination with Radiotherapy

In one example, orthotopic and/or subcutaneous tumor models in mice will be treated orally or by SQ injection with PCLX-001 in combination with radiotherapy.

Thus, in one example there is provided a method of forming an orthotopic solid tumor in a host, the method including the step of introducing cells into an orthotopic site in the host and allowing the introduced cells to form a tumor.

This example is useful, for example, in the production of tumors in animals than can be used to determine the efficacy of PCLX-001 as a radiosensitizer.

The term “orthotopic tumor” as used herein refers to any tumor formed in a particular organ in a host in which the cells in the tumor are derived from the same or similar cell type as that of the cells of the organ in which the tumor is present. For example, transformed cells from a mammary epithelial cell line may be introduced into mammary tissue in a host.

The term “orthotopic site” as used herein refers to a site in a host that has cells of the same or similar cell type as the cells being introduced.

In other examples, PCLX-001 in combination with radiotherapy may be evaluated in the context of an ectopic tumor model, or other suitable disease model.

In other examples, PCLX-001 may be evaluated with external radiotherapy to enhance tumor response.

In other examples, PCLX-001 may be evaluated with internal radiotherapy to enhance tumor response.

### Example 6—Investigations of PCLX-001's Ability to Cross the Blood-Brain Barrier in Orthotopic Brain Tumor Models in Combination with Radiotherapy

The term “blood brain barrier” as used herein refers to a selective partition, actively regulating the exchange of substances, including peptides, between the central nervous system (CNS) and the peripheral circulation.

In one example, PCLX-001 biodistribution within the CNS will be evaluated by assaying PCLX-001 in cerebral fluid, cerebral parenchyma and serum drug concentration post-drug administration in a normal animal model brain.

In one example, the animal model's normal brain will undergo radiation with a dose of 2, 4, 6 or 10 Gy followed by administration of PCLX-001 to determine biodistribution within the CNS will be evaluated by assaying PCLX-001 in cerebral fluid, cerebral parenchyma and serum drug concentration.

In one example, the biodistribution assay will be carried out with an orthotopic tumor implanted in the brain.

### Example 7—Demonstration of PCLX-001 (Referred to Herein as Zelenirstat) Radiosensitization In Vitro, Optimization of Dose and Schedule and Target Organs Informed by Human Plasma Pharmacokinetics and Murine Plasma, Brain, and Cerebrospinal Fluid Pharmacokinetics

PCLX-001 in Combination with Radiotherapy

Myristoylation is a form of protein lipidation, a co-translational and post-translational modification that involves the addition of a myristoyl group to the N-terminus of a protein. This modification is crucial for protein-protein interactions, protein stability, and subcellular localization (reference 1). PCLX-001 (referred to herein as zelenirstat) is a small molecule N-mryistoyltransferase (NMT) inhibitor that selectively kills human cancers, many of which have aberrant N-myristoyltransferase expression (references 2,3,4,5), by several described mechanisms (references 3,7,8,9). The mechanism of cytotoxic action is pleiotropic but one of its therapeutic effects is the inhibition of the src family kinases (SFK). PCLX-001 monotherapy has shown activity in multiple cancer types (references 3,4). Most cancer histologies tested with this drug respond to concentrations of drug in the nM range for IC50s and IC90s. PCLX-001 had not previously been studied in the context of combination with radiotherapy. However, there are indications that it may be a good radiosensitizer. PCLX-001's inhibition of NMT1 and NMT2 can lead to the downstream effects of poly(ADP-ribose) polymerase (PARP) cleavage and loss of function mediated through inhibiting calcium efflux from the endoplasmic reticulum. PARP cleavage in the context of radiotherapy can lead to delayed and unsuccessful DNA damage repair in response to radiation-induced DNA damage, thereby increasing the efficacy of radiotherapy.

Use and timing of combination therapies involving drugs and radiation therapy generally require an understanding of the plasma pharmacokinetics after administration. Furthermore, drug penetration to a target organ is a determinant of the utility of drugs used for radiosensitization. One such target organ is the brain, where many drugs are unable to penetrate the blood brain barrier in sufficient concentrations to permit radiosensitization of primary brain tumors or secondary brain tumors (the latter also known a brain metastases).

### Section I—Demonstration of Radiation Sensitization

### Materials and Methods:

### Culturing of Cancer Cells

Cancer cell lines of human origin were cultivated in the suitable culture medium enriched with 10% fetal bovine serum (FBS) and a 1% mixture of penicillin-streptomycin. These were incubated at a temperature of 37° C. in a 5% CO₂ humid environment. The cells were sub-cultured upon reaching a confluence of 80% with the aid of 0.25% trypsin-EDTA [Freshney, R. I., 2010. Culture of animal cells: a manual of basic technique and specialized applications. John Wiley & Sons].

### PCLX-001 Treatment

PCLX-001 was prepared at several concentrations for use as a radiosensitizing drug (serial dilutions from 1000 nM, including control 0 nM) in culture media. The cancer cells cultures were then treated with the aforementioned PCLX-001 concentrations and left for 24 h, 48 h, 72 h, and 96 h to allow for maximum pharmacodynamic effect. The cells were then plated into 96-well plates at 5000 cells per well and left to attach overnight while continuing to incubate in the drug/media cocktail.

### Application of Radiation

Upon completion of the PCLX-001 incubation period, a single radiation dose was administered to the cells using a preclinical irradiator. The quantity of radiation was decided based on the results from prior optimization tests [Hall, E. J., Giaccia, A. J., 2012. Radiobiology for the radiologist. Lippincott Williams & Wilkins.]. The radiation doses included 0, 2, and 4 Gy, which corresponds to the most common clinical radiation dose fractions used.

### Cell Viability Assessment

72 hours post-radiation, an Alamar Blue Assay was conducted to determine cell viability. Alamar Blue dye was added to the media (10% v/v) and the plates were incubated at 37° C. for 4 hours. Using a fluorescence plate reader, fluorescence was measured at excitation/emission wavelengths of 560/590 nm. To quantify cell viability, the results were normalized to the data from untreated controls [Ahmed, S. A., et al., 1994].

### Analysis of Data

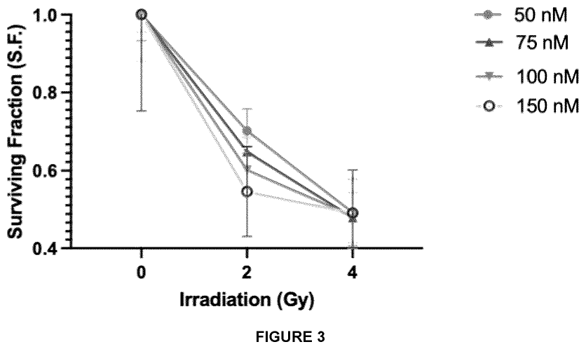

The data were shown as mean±standard deviation (SD) gathered from three or more independent tests. A one-way ANOVA followed by a post hoc Tukey test was used for statistical analyses to find significant disparities between treatment groups. The cut-off for statistical significance was a p-value of less than 0.05. A representative experiment is shown in FIG. 3.

### Results:

### PCLX-001 and Radiotherapy Delivery Schedule

Empirical testing suggests that the target timing window for administering PCLX-001 prior to radiotherapy delivery is about 72 hours, in order to provide substantive, up to maximal effectiveness in vitro. This is consistent with the observation that while PCLX-001 directly inhibits n-myristoylation within minutes of exposure to the cell, the residual N-myristoylated proteins often require 48 to 96 hours to show reduction in protein levels on Western blots, and the biological effects of PCLX-001 often become apparent only after 48 to 96 hours (reference 3). The kinetics of DNA damage repair, particularly double-strand breaks, are well known to occur as early as within the first 6 hours after irradiation. However, PCLX-001 manifests its downregulation in PARP levels and cleavage after a delayed period, typically after 3 days (reference 3). It appears that pre-treating cancer cells for 72 hours facilitates, if not substantially maximizes the synergistic nature of the drug and radiotherapy, presumably via this mechanism.

### Optimal Drug Dosing for Radio Sensitization

Through empirical testing, it was observed that PCLX-001 exhibited a radio sensitization effect in a drug dosing range of 50 nM to 150 nM. Below 25 nM, it was observed that the dose-enhancement factor in the tested glioblastoma cell line of U251 was below 1.2 (inadequate). Above 150 nM, it was observed that the toxicity of the drug itself exceeds the effect of typical radiotherapy dose-per-fraction's effect. This suggests the balance between drug toxicity and synergistic effect lies between 50 nM and 150 nM.

Within this range, statistically and clinically significant radiotherapy dose enhancement was observed at the typical 2 Gy per fraction radiotherapy dose seen in clinical treatment (see FIG. 3). Statistical significance is established via statistical analysis with a threshold for significance of p<0.05. Clinical significance, in the context of radiosensitizers, is commonly held as any drug that enhances radiation effect by 20% or greater. In U251 glioblastoma cell line, a dose enhancement factor of 1.8 to 2.0 was estimated based on these results, which constitutes significant radiosensitization effect in comparison to other clinically used radiosensitizers that range from 1.2 to 1.6. See FIG. 3.

Section II—PLCX-001 plasma pharmacokinetics determined in clinical trial NCT04836195 as the basis for selection of radiation timing, drug initiation timing, drug timing in relationship to radiation, and selection of drug dosage for radiation sensitization

### Materials and Methods:

### Plasma Pharmacokinetic Sampling, Assay, and Analyses

A phase I, multicenter, nonrandomized, open-label study of zelenirstat, entitled “Phase I Trial of PCLX-001 in B-cell Non-Hodgkin Lymphoma and Advanced Solid Malignancies” (reference 6), is being conducted and is registered at clinicaltrials.gov as NCT04836195. In this study, PCLX-001 was administered as an oral daily dose every morning to patients with advanced cancer, on 28-day cycles, until toxicity or progressive disease. Peripheral blood samples were collected over six time points in the first 8 hours of cycle 1 day 1, and again on cycle 1 day 15, with pretreatment day 1 levels obtained with every subsequent cycle. PCLX-001/zelenirstat plasma concentrations were quantified using a validated ultra-performance liquid chromatography with tandem mass spectrometry detection and analyzed using a nonlinear mixed-effects model.

### Results:

The full plasma pharmacokinetics of PCLX-001 across a broad array of doses has been investigated and are summarized in Table 1 below, as derived from 21 participants in study NCT04836195. In summary, following single oral administration of 20 mg to 210 mg PCLX-001/zelenirstat on Cycle 1, Day 1 (C1D1), it was observed that plasma concentrations of zelenirstat steadily increased reaching peak levels with median Tₘₐₓ values ranging from 1 to 4 hours across the dose cohorts. After repeated daily dosing, zelenirstat Tₘₐₓ values on C1D15 were comparable to values obtained on C1D1. The median Tₘₐₓ range was slightly wider (1 to 6 hours) in patients taking proton pump inhibitors (PPI) with a maximum Tₘₐₓ value observed of about 8 hours in those patients. After reaching peak concentrations (Cₘₐₓ) on C1D1, zelenirstat was eliminated from plasma with mean terminal half-life ranging from 6.7 to 9.5 hours across the dose cohorts.

Systemic exposure to zelenirstat (Cₘₐₓ and area under the concentration-time curve (AUC) values) tended to increase with increasing dose over the dose range of 20 mg to 210 mg. Dose-proportionality over this dose range was not supported by statistical analysis due to the limited number of subjects and the variability introduced by the concomitant use of proton pump inhibitors. The majority of patients had limited accumulation of zelenirstat (<2-fold) following repeated administration for 14-days, with accumulation ratios for Cₘₐₓ (Ar_{Cmax}) and AUC₀₋₂₄ (Ar_{AUC0-24}) values ranging from 0.41 to 1.72 and 1.0 to 1.9, respectively. Three subjects had accumulation ratios for both (Ar_{Cmax} and Ar_{AUC0-24}) between 3 and 6-fold. Based upon pre-dose concentrations over multiple cycles, no accumulation was apparent beyond Day 15. Time to steady state was assessed by visual observation of pre-dose zelenirstat concentrations. Steady state, with some variability, was achieved by day 8 to 15 of dose administration in most patients.

TABLE 1
Summary Statistics of Plasma Pharmacokinetic Parameters

| | | Number | | | AUC0-24 | |
|---|---|---|---|---|---|---|
| | | of | Tmax h | Cmax | (h * ng/ml) | t½ |
| Dose | | evaluable | (median, | (ng/ml) | (mean, | (h) |
| level | Timepoint | patients | range) | (mean, SD) | SD) | (mean, SD) |
| 20 mg | Cycle 1 D1 | 3 | 2.0 | 276 | 1583 | 6.7 |
| | | | (0.9-4.0) | (117) | (231) | (1.7) |
| | Cycle 1 | 3 | 1 | 359 | 2066 | 7.4 |
| | D15 | | (0.5-2.1) | (118) | (387) | (2.0) |
| 40 mg | Cycle 1 D1 | 3 | 0.9 | 848 | 4819 | 9.2 |
| | | | (0.5-3.9) | (831) | (4997) | (2.2) |
| | Cycle 1 | 3 | 2.2 | 774 | 7060 | 9.7 |
| | D15 | | (2.0-4.1) | (720) | (6711) | (3.0) |
| 70 mg | Cycle 1 D1 | 3 | 3.9 | 665 | 5654 | 9.5 |
| | | | (1.0-7.7) | (675) | (5232) | (NC) |
| | Cycle 1 | 3 | 4.1 | 467 | 6613 | NC |
| | D15 | | (4.0-7.9) | (257) | (3526) | |
| 100 mg | Cycle 1 D1 | 5 | 2.0 | 2188 | 18936 | 8.0 |
| | | | (2.0-3.8) | (1037) | (9188) | (2.3) |
| | Cycle 1 | 3 | 4.0 | 2300 | 24714 | 12.0 |
| | D15 | | (2.0-4.0) | (1664) | (14020) | (NC) |
| 140 mg | Cycle 1 D1 | 3 | 1.0 | 1769 | 11883 | 7.2 |
| | | | (0.5-1.1) | (1208) | (8823) | (0.9) |
| | Cycle 1 | 3 | 2.0 | 1335 | 8150 | 10.4 |
| | D15 | | (1.0-2.0) | (964) | (2846) | (5.5) |
| 210 mg | Cycle 1 D1 | 4 | 3.0 | 1767 | 14654 | 7.8 |
| | | | (1.9-4.0) | (757) | (6715) | (2.4) |
| | Cycle 1 | 4 | 2.0 | 2935 | 27558 | 7.7 |
| | D15 | | (1.0-3.9) | (1047) | (13349) | (3.4) |

Abbreviations:
AUC0-24, area under the plasma concentration-time curve; Cmax, maximum concentration; T½, half-life; Tmax, time to maximum serum concentration; PK = pharmacokinetic. N = number of participants in the PK analysis set, n = number of participants. SD = standard deviation. NC = not calculated. NA = not available

### Section III—Determination of PCLX-001 Brain Penetration and Accumulation Through the Blood Brain Barrier

### Background:

The blood-brain barrier (BBB) is a highly selective semipermeable border of endothelial cells that prevents solutes in the circulating blood from non-selectively crossing into the central nervous system's extracellular fluid (CSF) while still allowing for the diffusion of essential substances like glucose and oxygen. The BBB is formed by brain capillary endothelial cells which are connected by tight junctions, a type of intercellular junction that forms a virtually impermeable barrier to fluid. These cells are surrounded and supported by pericytes and astrocyte end-feet. The blood-brain barrier is important to protecting the brain from harmful substances in the blood, maintaining homeostasis, and regulating the transport of necessary molecules into the brain. Its selectivity is needed to support effective neuronal function and protects the brain from many common bacterial infections.

However, the BBB can also obstruct the delivery of therapeutic drugs to the brain, which is a major challenge in the treatment of many neurological disorders, such as brain tumors and neurodegenerative diseases. Consequently, much research is being done to develop methods to bypass or temporarily open the BBB for therapeutic purposes (reference Pardridge, W. M., 2005. The blood-brain barrier: bottleneck in brain drug development. NeuroRx, 2(1), pp. 3-14).

Computer modeling and drug exposure/brain penetration and accumulation studies were conducted to determine whether PCLX-001 accumulated in brain tissue.

### Materials and Methods:

### Computer Modeling Simulation of PCLX-001 BBB Penetration:

Support vector machine (SVM) pubchemFP modeling software (https://pubchem.ncbi.nlm.nih.gov) predicted a BBB penetration score of 0.094 for PCLX-001. This exceeds the threshold of 0.00 for crossing the BBB, where a negative value indicates a compound will not cross the BBB and a more positive value indicates a higher propensity to cross the BBB. PCLX-001 is therefore expected to penetrate the BBB. For comparison, temozolomide, the most commonly used radiosensitizer and anticancer agent for primary brain tumors, is predicted to have a lower BBB penetration score of 0.034, suggesting that PCLX-001 may exhibit better brain penetration than the commercial product in this space.

### Measured Brain Tissue PCLX-001 Accumulation:

The pharmacokinetic parameters and tissue accumulation of PCLX-001 were determined in male C57BL/6 mice. All procedures were performed following relevant guidelines and regulations, and ethical approval was obtained from the Institutional Animal Care and Use Committee.

### Animals and Drug Administration:

Male C57BL/6 mice aged 8-10 weeks were used for the study. The mice were acclimated for one week prior to the start of the experiment with free access to food and water. The drugs PCLX-001 were administered at 5 mg/kg body weight, either intravenously (IV) through the tail vein or orally by gavage (OG). For IV and OG administration, the drugs were diluted in sterile saline and for OG, they were suspended in sterile water.

### Sampling:

At predetermined time points post-administration (0.083, 0.25, 0.5, 1, 2, 4, 8, 24 hours), blood samples were collected into heparinized tubes and immediately centrifuged at 4° C. to separate the plasma. Mice were then sacrificed by cervical dislocation while under anesthesia, and cerebrospinal fluid (CSF) was collected. The brain was quickly excised, rinsed with cold saline to remove residual blood, blotted dry, weighed, and snap-frozen in liquid nitrogen. All samples were stored at −80° C. until analysis.

### Sample Analysis:

The concentrations of PCLX-001 in the plasma, CSF, and brain tissue were determined by a validated liquid chromatography-tandem mass spectrometry (LC-MS/MS) method. Brain tissues were homogenized before analysis, and all samples were processed with an appropriate extraction procedure to isolate the drugs.

### Pharmacokinetic and Tissue Drug Level Analysis:

Pharmacokinetic parameters including Cₘₐₓ (maximum observed drug concentration), Tₘₐₓ (time to reach Cₘₐₓ), AUC (area under the concentration-time curve), T_{1/2} (elimination half-life), and the bioavailability for both IV and OG administration were determined by non-compartmental analysis using standard pharmacokinetic software. The ratios of drug concentration in the brain and CSF to plasma were calculated to evaluate the ability of PCLX-001 to cross the blood-brain barrier.

### Results:

It was observed: (I) that oral administration of PCLX-001 in a single dose exhibited a relatively short Tₘₐₓ and a relatively high % F (where % F indicates systemic exposure after oral absorption of drug is essentially equivalent to intravenous tail vein injection of drug), (II) that PCLX-001 rapidly accumulated in brain tissue, and (III) exhibited radiation sensitization effects at concentrations between about 50 nM-150 nM, as demonstrated in Section I. The measured brain concentrations over time suggested peak brain drug concentrations occurred approximately 4 h after oral administration. The tissue to plasma ratio was found to be 0.02, indicating relatively significant accumulation in brain tissue. The drug retention in brain may be longer than in plasma, with half-life of 4.17 hours rather than 1.77 hours in plasma. These data are show in Tables 2 and 3.

TABLE 2
Concentration time data of PCLX-001 after 5 mg/kg oral administration

| Animal | Time point | Plasma Conc. | Mean Plasma | Brain Conc. | Mean Brain | CSF Conc. | Mean CSF | | Brain/ |
|---|---|---|---|---|---|---|---|---|---|
| ID | (h) | (nM) | Conc. (n.M) | (nM) | Conc. (n.M) | (nM) | Conc. (DNI) | B/P-Kp | CSF-Kp |
| 25 | 0.00 | 0.00 | 0.00 | 0.00 | 0.00 | 0.00 | 0.00 | NA | NA |
| 26 | | 0.00 | | 0.00 | | 0.00 | | | |
| 27 | | 0.00 | | 0.00 | | 0.00 | | | |
| 28 | 0.25 | 8570.40 | 8308.70 | 86.13 | 98.35 | 7.47 | 7.58 | 0.01 | 12.97 |
| 29 | | 9593.14 | | 135.18 | | 11.50 | | | |
| 30 | | 6762.56 | | 73.74 | | 3.78 | | | |
| 31 | 0.50 | 4654.05 | 6513.39 | 107.73 | 122.82 | 3.44 | 3.51 | 0.02 | 35.02 |
| 32 | | 8294.21 | | 122.31 | | 4.19 | | | |
| 33 | | 6591.89 | | 138.42 | | 2.89 | | | |
| 34 | 1.00 | 7236.04 | 8390.34 | 75.45 | 64.80 | 2.72 | 4.96 | 0.01 | 13.06 |
| 35 | | 9051.65 | | 54.48 | | 8.67 | | | |
| 36 | | 8883.32 | | 64.47 | | 3.50 | | | |
| 37 | 2.00 | 9734.69 | 7039.39 | 185.79 | 157.33 | 7.26 | 13.23 | 0.02 | 11.89 |
| 38 | | 5724.05 | | 81.69 | | 15.58 | | | |
| 39 | | 5659.43 | | 204.51 | | 16.86 | | | |
| 40 | 4.00 | 13975.97 | 10625.84 | 243.03 | 176.29 | 15.26 | 36.64 | 0.02 | 4.81 |
| 4 ? | | 6234.47 | | 114.75 | | 6.67 | | | |
| 42 | | 11667.07 | | 171.09 | | 87.98 | | | |
| 43 | 8.00 | 2821.68 | 5409.32 | 79.32 | 120.98 | 30.28 | 14.48 | 0.02 | 8.35 |
| 44 | | 6573.92 | | 135.06 | | 5.97 | | | |
| 45 | | 6832.35 | | 148.56 | | 7.19 | | | |
| 46 | 24.00 | 6.39 | 5.63 | 0.00 | 6.98 | 0.00 | 0.00 | 1.24 | NA |
| 47 | | 6.38 | | 8.13 | | 0.00 | | | |
| 48 | | 4.10 | | 12.81 | | 0.00 | | | |

B/P- brain to plasma ratio, B/CSF- Brain/CSF

TABLE 3
Summary statistics of PCLX-001 plasma, brain, and CSF pharmacokinetics

| | | | | C0/Cₘₐₓ | AUC₀₋ₗₐₛₜ | T½ | | T/P |
|---|---|---|---|---|---|---|---|---|
| Conditions | Analyte | Matrix | Tₘₐₓ (h) | (nM) | (nM * h) | (h) | % F | ratio |
| PCLX-001 oral | PCLX-001 | Plasma | 4 | 10625.84 | 107887.8 | 1.77 | 97.00 | NA |
| gavage 5 mg/kg | | Brain | 4 | 176.29 | 2149.75 | 4.17 | ND | 0.02 |
| | | CSF | 4 | 36.64 | 165.65 | NR | | |

C0- Back extrapolated concentration at time zero for IV for plasma, T/P- Tissue to plasma AUClast ratio; ND- Not Determined; NA- not applicable; NR

### Discussion of Findings in Sections I, II, and III

The experimental details described in foregoing sections I-III indicate the utility of PCLX-001 as a radiosensitizer, and in aggregate, may provide insights informing the design of pre-clinical and clinical trials and clinical care of patients with cancer, combining PCLX-001 with radiotherapy to treat cancer.

The above-delineated pre-clinical testing indicated that PCLX-001 was an effective radiosensitizer in the cultured cancer cells tested. The model cell line used was a human Glioblastoma cell line commonly used in such studies, demonstrating a robust radiation sensitization effect. Testing suggested that the drug delivery timing is about 72 hours prior to radiotherapy as an induction regimen, to allow for the needed downregulation for DNA-damage repairing proteins, followed by daily dosing to maintain suppression of these proteins. The highest therapeutic index as a radiosensitizer was estimated to lie in the 50 nM to 150 nM drug dosing range. In the cancer cell line tested, the addition of PCLX-001 appeared to increase radiotherapy's effectiveness by about 80%.

Combining these radiosensitization effects with the full pharmacokinetic properties across a broad range of daily oral PCLX-001 doses lead to several conclusions that inform the design of combination PCLX-001+radiation regimens, in terms of timing, drug dosing, and scheduling of the onset of radiation with relation to initiation of daily oral PCLX-001. For example, several components of a clinical administration schedule may be drawn from the data, including:
i) pre-treating patients with oral PCLX-001 before radiation, rather than the converse.
ii) pre-treating the patient with oral PCLX-001 for 3 to 5 daily doses before beginning radiation, in order to achieve steady state drug levels and pharmacodynamic effects in the target tissues.
iii) delivering daily radiation therapy at the time of peak plasma concentrations of drug, for example two hours after oral PCLX-001.
iv) discontinuing patients from proton pump inhibitors prior to combination of oral PCLX-001 and radiation therapy, in order to have a predictable peak concentration
v) selecting a dose of PCLX-001 that achieves concentrations of 50 nM to 100 nM in the target tissue at the time of radiation therapy.
vi) using lower doses of PCLX-001 for combination radiation and PCLX-001 therapy, to reduce systemic toxicity while preserving radiosensitization, given that the above-noted target concentrations are lower than that achieved with higher doses of PCLX-001.
vii) treating primary brain tumors or secondary brain tumors with a combination of oral PCLX-001 followed by radiotherapy four hours later.

The foregoing results provided foundational insights into the way to administer PCLX-001 with radiation to increase the therapeutic index of the combination therapy, and improve the benefit to risk ratio of such combination therapies.

When reducing these learnings to practice, for one example, it might be appropriate to treat a radiation patient with a non-brain tumor regimen that gives PCLX-001 daily by mouth beginning three days prior to initiation of daily fractions of external beam radiotherapy, timed to follow 2 hours after each daily dose of PCLX-001, and select a non-toxic drug dosage of 100 mg rather than the higher, more toxic, anticancer drug dosages.

For another example, it might be appropriate to treat a radiation patient with a primary brain tumor or a secondary brain tumor with a regimen that gives PCLX-001 daily by mouth beginning three days prior to initiation of daily fractions of external beam radiotherapy, timed to follow 4 hours after each daily dose of PCLX-001, and select a non-toxic drug dosage of 100 mg rather than the higher, more toxic, anticancer drug dosages.

### REFERENCES

1. Castrec, B., Dian, C., Ciccone, S., Ebert, C. L., Bienvenut, W. V., Le Caer, J. P., Steyaert, J. M., Giglione, C., and Meinnel, T. (2018). Structural and genomic decoding of human and plant myristoylomes reveals a definitive recognition pattern. Nat Chem Biol 14, 671-679.
2. Selvakumar, P., Lakshmikuttyamma, A., Shrivastav, A., Das, S. B., Dimmock, J. R., and Sharma, R. K. (2007). Potential role of N-myristoyltransferase in cancer. Prog Lipid Res 46, 1-36.
3. Beauchamp, E., Yap, M. C., Iyer, A., Perinpanayagam, M. A., Gamma, J. M., Vincent, K. M., Lakshmanan, M., Raju, A., Tergaonkar, V., Tan, S. Y., et al. (2020). Targeting N-myristoylation for therapy of B-cell lymphomas. Nat Commun 11, 5348.
4. Mackey, J. R., Lai, J., Chauhan, U., Beauchamp, E., Dong, W. F., Glubrecht, D., Sim, Y. W., Ghosh, S., Bigras, G., Lai, R., et al. (2021). N-myristoyltransferase proteins in breast cancer: prognostic relevance and validation as a new drug target. Breast Cancer Res Treat 186, 79-87.
5. Weickert, M., Dillberger, J., Mackey, J. R., Wyatt, P., Gray, D., Read, K., Li, C., Parenteau, A., and Berthiaume, L. G. (2019). Initial Characterization and Toxicology of an Nmt Inhibitor in Development for Hematologic Malignancies. Blood 134, 3362-3362.
6. Sangha, R., Mackey, J. R., Sehn, L. H., Kuruvilla, J., Weickert, M. J., and Berthiaume, L. G. (2021). An Open-Label, First-in-Human, Phase I Trial of Daily PCLX-001. Blood 138, 1364-1364.
7. Erwan Beauchamp, Chistopher Cromwell, Eman Moussa, Aishwarya Iyer, Megan Yap, Rony Pain, Jay Gamma, Olivier Julien, Basil Hubbard, Luc Berthiaume. Understanding the sensitivity of cancer cells to myristoylation inhibitors for oncology applications [abstract]. In: Proceedings of the American Association for Cancer Research Annual Meeting 2023; Part 1 (Regular and Invited Abstracts); 2023 Apr. 14-19; Orlando, FL. Philadelphia (PA): AACR; Cancer Res 2023; 83(7_Suppl):Abstract nr 1662.
8. Erwan Beauchamp, Jay Gamma, Christopher R. Cromwell, Eman W. Moussa, Aishwarya Iyer, Megan Yap, Rony Pain, Morris Kostiuk, Krista M. Vincent, Lynne M. Postovit, Olivier Julien, Basil P. Hubbard, John R. Mackey & Luc G. Berthiaume. Myristoylation inhibition impacts oxidative phosphorylation and defines a 91 gene sensitivity signature that identifies new solid cancer indications. Manuscript submitted.
9. Rony Pain, Erwan Beauchamp, Katia Carmine-Simmen, Jay Gamma, Rebecca Reif, Abul Azad, Allan Murray, John Lewis, Luc Berthiaume; Abstract 3620: N-myristoylation inhibition reduces angiogenesis and cancer cell migration. Cancer Res 1 Apr. 2023; 83 (7_Supplement): 3620. https://doi.org/10.1158/1538-7445.AM2023-3620

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed is:

1. Use of PCLX-001, or pharmaceutically acceptable salt thereof, for radiosensitizing a cancerous cell in a subject in need of radiation therapy.

2. Use of PCLX-001, or pharmaceutically acceptable salt thereof, in the manufacture of a medicament for radiosensitizing a cancerous cell in a subject in need of radiation therapy.

3. The use of claim 1 or 2, wherein said radiation therapy is external radiation therapy, internal radiation therapy or systemic radiation therapy.

4. The use of any one of claims 1 to 3, wherein said subject has cancer of the bladder, brain, breast, cervix, larynx, lung, prostate, vagina, thyroid, pancreas, ovary, breast, uterus, gallbladder, perianal and pelvic regions; colorectal cancers; gynecological cancers; cancer of the small intestine; small cell lung cancer; head and neck cancer; bronchial cancer; oral cancer; rectal cancer; tracheal cancer; or adult non-Hodgkin lymphoma.

5. The use of claim 4, wherein the brain cancer is an astrocytoma, glioma, embryonal tumour, non-malignant brain tumor, pediatric brain tumour, or metastatic tumours.

6. The use of claim 5, wherein the astrocytoma is anaplastic astrocytoma, diffuse astrocytoma, pilocytic astrocytoma, or glioblastoma.

7. The use of claim 5, wherein the glioma is diffuse midline glioma, oligodendroglioma, ependymoma, or optic pathway glioma.

8. The use of claim 5, wherein the embryonal tumour is atypical teratoid/rhaboid (AT/RT), or medulloblastoma.

9. The use of claim 5, wherein the non-malignant brain tumour is acoustic neuroma, meningioma, pituitary adenomas, craniopharyngioma, or pilocytic astrocytoma.

10. The use of claim 5, wherein the pediatric brain tumour is atypical teratoid/rhaboid tumour (AT/RT), diffuse midline glioma, medulloblastoma, pilocytic astrocytoma, craniopharyngioma, ependymoma, or optic pathway glioma.

11. The use of any one of claims 1 to 10, wherein said subject is a human.

12. The use of any one of claims 1 to 11, wherein the compound PCLX-001 or pharmaceutically acceptable salt thereof is at a dosing range of about 50 nM to about 150 nM.

13. Use of a radiosensitizer, wherein the radiosensitizer is PCLX-001, and use of radiation therapy, for treating a subject with cancer, suspected of having a cancer, or at risk of developing a cancer.

14. Use of a radiosensitizer, wherein the radiosensitizer is PCLX-001, in the manufacture of a medicament, and use of radiation therapy, for treating a subject with cancer, suspected of having a cancer, or at risk of developing a cancer.

15. The use of claim 13, or 14, wherein said radiation therapy is external radiation therapy, internal radiation therapy or systemic radiation therapy.

16. The use of any one of claims 13 to 15, wherein the said subject has cancer of the bladder, brain, breast, cervix, larynx, lung, prostate, vagina, thyroid, pancreas, ovary, breast, uterus, gallbladder, perianal and pelvic regions; colorectal cancers; gynecological cancers; cancer of the small intestine; small cell lung cancer; head and neck cancer; bronchial cancer; oral cancer; rectal cancer; tracheal cancer; or adult non-Hodgkin lymphoma.

17. The use of claim 16, wherein the brain cancer is an astrocytoma, glioma, embryonal tumour, non-malignant brain tumor, pediatric brain tumour, or metastatic tumours.

18. The use of claim 17, wherein the astrocytoma is anaplastic astrocytoma, diffuse astrocytoma, pilocytic astrocytoma, or glioblastoma.

19. The use of claim 17, wherein the glioma is diffuse midline glioma, oligodendroglioma, ependymoma, or optic pathway glioma.

20. The use of claim 17, wherein the embryonal tumour is atypical teratoid/rhaboid (AT/RT), or medulloblastoma.

21. The use of claim 17, wherein the non-malignant brain tumour is acoustic neuroma, meningioma, pituitary adenomas, craniopharyngioma, or pilocytic astrocytoma.

22. The use of claim 17, wherein the pediatric brain tumour is atypical teratoid/rhaboid tumour (AT/RT), diffuse midline glioma, medulloblastoma, pilocytic astrocytoma, craniopharyngioma, ependymoma, or optic pathway glioma.

23. The use of any one of claims 13 to 22, further comprising use or one or more drugs for treating brain cancer comprising: conjugates of methotrexate, vinca alkaloids, carmustine, cisplatin, carboplatin, nitrosourea, hydroxyurea, cyclophosphamide, etoposide, procarbazine, irinotecan, lomustine, vincristine, and/or temozolomide.

24. The use of any one of claims 13 to 22, further comprising use of one or more drugs in the manufacture of a medicament for treating brain cancer comprising: conjugates of methotrexate, vinca alkaloids, carmustine, cisplatin, carboplatin, nitrosourea, hydroxyurea, cyclophosphamide, etoposide, procarbazine, irinotecan, lomustine, vincristine, and/or temozolomide.

25. The use of any one of claims 13 to 24, wherein said subject is a human.

26. The use of any one of claims 13 to 23, comprising use of the radiosensitizer at a dosing to achieve blood or target organ or tumor drug concentrations in the range of about 50 nM to about 150 nM.

27. The use of any one of claims 13 to 26, comprising use of the radiosensitizer at Time A and use of radiation therapy at Time B for achieving a target dosing range in blood or target organ or tumor drug concentration of about 50 nM to about 150 nM at the cancer site, or at the potential cancer site.

28. The use of claim 27, wherein Time B is about 2 hours to about 4 hours after Time A when administering the radiosensitizer at Time A comprises orally administering the radiosensitizer.

29. The use of claim 27, wherein Time B is about 72 hours after Time A when administering the radiosensitizer at Time A comprises commencing radiosensitizer therapy.

30. The use of any one of claims 1 to 29, wherein administering PCLX-001 comprises PCLX-001 crossing the blood brain barrier.

31. The use of claim 30, wherein use of PCLX-001 comprises PCLX-001 crossing the blood brain barrier and acting as a radiosensitizer for primary brain tumors.

32. The use of claim 30 or 31, wherein use of PCLX-001 comprises PCLX-001 crossing the blood brain barrier and acting as a radiosensitizer for primary brain tumors about 4 hours after administration.

33. The use of any one of claims 30 to 32, wherein use of PCLX-001 comprises PCLX-001 crossing the blood brain barrier and acting as a radiosensitizer for secondary brain tumors.

34. The use of any one of claims 30 to 33, wherein use of PCLX-001 comprises PCLX-001 crossing the blood brain barrier and acting as a radiosensitizer for secondary brain tumors about 4 hours after administration.

35. The use of any one of claims 13 to 34, wherein the patient discontinues use of a proton pump inhibitor prior to treatment with the radiosensitizer and radiation therapy, if the patient is using the proton pump inhibitor.

36. A method of radiosensitizing a cancerous cell in a subject in need of radiation therapy, comprising: administering to a subject compound PCLX-001, or pharmaceutically acceptable salt thereof.

37. The method of claim 36, wherein said radiation therapy is external radiation therapy, internal radiation therapy or systemic radiation therapy.

38. The method of claim 36 or 37, wherein said subject has cancer of the bladder, brain, breast, cervix, larynx, lung, prostate, vagina, thyroid, pancreas, ovary, breast, uterus, gallbladder, perianal and pelvic regions; colorectal cancers; gynecological cancers; cancer of the small intestine; small cell lung cancer; head and neck cancer; bronchial cancer; oral cancer; rectal cancer; tracheal cancer; or adult non-Hodgkin lymphoma.

39. The method of claim 38, wherein the brain cancer is an astrocytoma, glioma, embryonal tumour, non-malignant brain tumor, pediatric brain tumour, or metastatic tumours.

40. The method of claim 39, wherein the astrocytoma is anaplastic astrocytoma, diffuse astrocytoma, pilocytic astrocytoma, or glioblastoma.

41. The method of claim 39, wherein the glioma is diffuse midline glioma, oligodendroglioma, ependymoma, or optic pathway glioma.

42. The method of claim 39, wherein the embryonal tumour is atypical teratoid/rhaboid (AT/RT), or medulloblastoma.

43. The method of claim 39, wherein the non-malignant brain tumour is acoustic neuroma, meningioma, pituitary adenomas, craniopharyngioma, or pilocytic astrocytoma.

44. The method of claim 39, wherein the pediatric brain tumour is atypical teratoid/rhaboid tumour (AT/RT), diffuse midline glioma, medulloblastoma, pilocytic astrocytoma, craniopharyngioma, ependymoma, or optic pathway glioma.

45. The method of any one of claims 36 to 44, wherein said subject is a human.

46. The method of any one of claims 36 to 45, comprising administering the compound PCLX-001 at a dosing range of about 50 nM to about 150 nM.

47. A method of treating a subject with cancer, suspected of having a cancer, or at risk of developing a cancer, comprising administering a radiosensitizer, wherein the radiosensitizer is PCLX-001, and administering radiation therapy.

48. The method of claim 47, wherein said radiation therapy is external radiation therapy, internal radiation therapy or systemic radiation therapy.

49. The method of claim 47 or 48, wherein the said subject has cancer of the bladder, brain, breast, cervix, larynx, lung, prostate, vagina, thyroid, pancreas, ovary, breast, uterus, gallbladder, perianal and pelvic regions; colorectal cancers; gynecological cancers; cancer of the small intestine; small cell lung cancer; head and neck cancer; bronchial cancer; oral cancer; rectal cancer; tracheal cancer; or adult non-Hodgkin lymphoma.

50. The method of claim 49, wherein the brain cancer is an astrocytoma, glioma, embryonal tumour, non-malignant brain tumor, pediatric brain tumour, or metastatic tumours.

51. The method of claim 50, wherein the astrocytoma is anaplastic astrocytoma, diffuse astrocytoma, pilocytic astrocytoma, or glioblastoma.

52. The method of claim 50, wherein the glioma is diffuse midline glioma, oligodendroglioma, ependymoma, or optic pathway glioma.

53. The method of claim 50, wherein the embryonal tumour is atypical teratoid/rhaboid (AT/RT), or medulloblastoma.

54. The method of claim 50, wherein the non-malignant brain tumour is acoustic neuroma, meningioma, pituitary adenomas, craniopharyngioma, or pilocytic astrocytoma.

55. The method of claim 50, wherein the pediatric brain tumour is atypical teratoid/rhaboid tumour (AT/RT), diffuse midline glioma, medulloblastoma, pilocytic astrocytoma, craniopharyngioma, ependymoma, or optic pathway glioma.

56. The method of any one of claims 48 to 55, further comprising administering one or more drugs for treating brain cancer comprising: conjugates of methotrexate, vinca alkaloids, carmustine, cisplatin, carboplatin, nitrosourea, hydroxyurea, cyclophosphamide, etoposide, procarbazine, irinotecan, lomustine, vincristine, and/or temozolomide.

57. The method of any one of claims 48 to 56, wherein said subject is a human.

58. The method of any one of claims 48 to 57, comprising administering the radiosensitizer at a dosing to achieve blood or target organ or tumor drug concentrations in the range of about 50 nM to about 150 nM.

59. The method of any one of claims 48 to 58, comprising administering the radiosensitizer at Time A and administering radiation therapy at Time B for achieving a target dosing range in blood or target organ or tumor drug concentration of about 50 nM to about 150 nM at the cancer site, or at the potential cancer site.

60. The method of claim 59, wherein Time B is about 2 hours to about 4 hours after Time A when administering the radiosensitizer at Time A comprises orally administering the radiosensitizer.

61. The method of claim 59, wherein Time B is about 72 hours after Time A when administering the radiosensitizer at Time A comprises commencing radiosensitizer therapy.

62. The method of any one of claims 36 to 61, wherein administering PCLX-001 comprises PCLX-001 crossing the blood brain barrier.

63. The method of claim 62, wherein administering PCLX-001 comprises PCLX-001 crossing the blood brain barrier and acting as a radiosensitizer for primary brain tumors.

64. The method of claim 62 or 63, wherein administering PCLX-001 comprises PCLX-001 crossing the blood brain barrier and acting as a radiosensitizer for primary brain tumors about 4 hours after administration.

65. The method of any one of claims 62 to 64, wherein administering PCLX-001 comprises PCLX-001 crossing the blood brain barrier and acting as a radiosensitizer for secondary brain tumors.

66. The method of any one of claims 26 to 65, wherein administering PCLX-001 comprises PCLX-001 crossing the blood brain barrier and acting as a radiosensitizer for secondary brain tumors about 4 hours after administration.

67. The method of any one of claims 47 to 66, wherein the patient discontinues use of a proton pump inhibitor prior to treatment with the radiosensitizer and radiation therapy, if the patient is using the proton pump inhibitor.
